SEQUENCE LISTING (4 sequences)

SEQ ID NO: 1 - DNA, 189 bp, Cricket paralysis virus
aaaatgtgatcttgcttgtaaatacaattttgagaggttaataaattacaagtagtgctatttttgtatttaggttagctatttagctttacgttccaggatgcctagtggcagccccacaatatccaggaagccctctctgcggtttttcagattaggtagtcgaaaaacctaagaaatttacctgct

SEQ ID NO: 2 - DNA, 462 bp, Foot-and-mouth disease virus
tgcaggtagccccaactgacacaaaccgtgcaacttggaaccccgcctgggctttccaggtctagaggggtgacgccttgtactgtgtttgactccacgctcggtccactagcgagtgttagtagtagtactgttgcttcgtagcggagcatgacggccgtgggaatccctccttggcaacaaggacccacggggccgaaagccacgtcctgaaggacccgtcatgtgtgcaaccccagcacggcagctttattatgaaacccactttaaggtgacactgatactggtactcaaacactggtgacaggctaaggatgcccttcaggtaccccgaggtaacacgcgacactcgggatctgagaaggggactggggcttctataaaagtgcccagtttaaaaagcttctatgcctggataggcgaccggaggccggcgcctttcctttgaccactactgtttac

SEQ ID NO: 3 - DNA, 363 bp, Flavivirus FSME
gttagctctttctcgtatacgatattggatacactaaatttcgatttgctctagggcacccctccagcgacggccgaaatgggctagccatgcccatagtaggactagcaaacggagggactagccgtagtggcgagctccctgggtggtctaagtcctgagtacaggacagtcgtcagtagttcgacgtgagcactrgcccacctcgagatgctacgtggacgagggcatgcccaagacacaccttaaccctggcgggggtcgctagggtgaaatcacattatgtgatgggggtacgacctgatagggtgctgcagaggcccactagcaggctagtataaaaatctctgctgtacatggcac

SEQ ID NO: 4 - DNA, 597 bp, Artificial Sequence
  Based uppon CrPV
gggagaccctcgaatggaattggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatgctaccgtttaatattgcgtcaggtagtaaactactaactacaacctgctgaagcaaaaatgtgatcttgcttgtaaatacaattttgagaggttaataaattacaagtagtgctatttttgtatttaggttagctatttagctttacgttccaggatgcctagtggcagccccacaatatccaggaagccctctctgcggtttttcagattaggtagtcgaaaaacctaagaaatttacctgctggtagtaaactactaactacaacctgctgaagcagatgttttcttgggttaattgaggcctgagtataaggtgacttatacttgtaatctatctaaacggggaacctctctagtagacaatcccgtgctaaattgtaggactaattccatttatcagatttctag

(19) United States
(12) Patent Application Publication
(10) Pub. No.: US 2021/0222178 A1
(43) Pub. Date: Jul. 22, 2021

(54) MICROBIAL SYSTEM FOR PRODUCTION AND DELIVERY OF EUKARYOTE-TRANSLATABLE mRNA TO EUKARYA

(71) Applicant: SiVEC Biotechnologies LLC, Fort Collins, CO (US)

(72) Inventors: Lyndsey M. Linke, Fort Collins, CO (US); Ashley B. Williams, Austin, TX (US); Darcy Mora, Fort Collins, CO (US); Madeline Cox, Laporte, CO (US)

(73) Assignee: SiVEC Biotechnologies LLC, Fort Collins, CO (US)

(21) Appl. No.: 17/146,391

(22) Filed: Jan. 11, 2021

Related U.S. Application Data
(60) Provisional application No. 63/118,593, filed on Nov. 25, 2020.
(60) Provisional application No. 62/959,976, filed on Jan. 11, 2020.

(51) Int. Cl. C12N 15/70; A61K 35/74

(52) U.S. Cl. CPC C12N 15/70; A61K 35/74; C12N 2840/203; C12N 2840/60; C12N 2840/10; C12N 2800/101; C12N 2830/50

(57) ABSTRACT

A bacterial system for the generation and delivery of eukaryote-translatable mRNA to eukaryotic cells. The system uses invasive, non-pathogenic bacteria to generate and deliver functional mRNA cargo to eukaryotic cells. Additionally, the system uses bacteria to generate functional mRNA that can be extracted from the bacterial cell for downstream applications. The bacteria contain at least one prokaryotic expression cassette encoding the mRNA; the mRNA contains a bacterially transcribed poly-A sequence, and a 5′ cap or pseudo-cap element, e.g., an internal ribosome entry site (IRES) element, that will mediate translation in the eukaryotic host cell. Examples of therapeutic mRNA function include, but are not limited to, providing genetic material encoding antibodies, vaccine antigens, and defective genes in the host.

### CROSS-REFERENCE TO RELATED APPLICATIONS

This application claims the benefit of U.S. Provisional Application No. 62/959,976, filed Jan. 11, 2020 and U.S. Provisional Application No. 63/118,593, filed Nov. 25, 2020.

### REFERENCE TO AN ELECTRONIC SEQUENCE LISTING

The contents of the electronic sequence listing (SiVEC-Sequences-0111-03-US1.txt; Size: 2,908 bytes; Date of Creation: Feb. 2, 2021) is herein incorporated by reference in its entirety.

### FIELD OF INVENTION

This invention relates the production of messenger ribonucleic acid (mRNA). More specifically, this invention relates to a prokaryotic expression system to generate mRNAs within a bacterium which can further be utilized as a bacterial delivery vehicle for delivery to a eukaryotic host cell and immediate translation into a protein.

### BACKGROUND OF THE INVENTION

Cells use messenger RNA (mRNA) to translate information encoded in a cell's DNA into proteins. Since mRNA can encode any protein, this nucleic acid has the potential to be used therapeutically. In one such scenario, an exogenous mRNA is delivered to host cells and translated into one or more proteins, including enzymes, antibodies, and antigens that can function in a wide range of therapeutic applications. However, exogenous mRNA must be delivered safely, efficiently, and as a molecule capable of translation into a protein. Currently, no system exists that encompasses both the generation and the safe and efficacious delivery of fully translatable mRNAs without integration into the host genome for proper processing. mRNA can also be produced using a microbial system. In this scenario, exogenous mRNA is produced inside a bacterial cell and collected from the bacterial cell for downstream translation into proteins, including enzymes, antibodies, and antigens that can function in a wide range of therapeutic and non-therapeutic applications inside a eukaryotic cell. However, exogenously produced (i.e., bacterially produced) mRNA must be a molecule capable of eukaryotic translation into a protein. Currently, no complete system exists that encompasses the generation of eukaryote-translatable mRNAs inside a bacterial cell without post-transcriptional processing in a test tube or integration into the host genome for proper processing. Herein, eukaryote-translatable mRNA means mRNA that contains required elements on the 5′- and 3′-ends that support the translation of the mRNA into a protein inside a eukaryotic cell.

### SUMMARY OF THE INVENTION

The present invention provides a bacterial system for the scalable microbial biomanufacturing (also referred to as production or generation) of eukaryote-translatable mRNA and in some cases where desired, the subsequent intracellular delivery of the eukaryote-translatable mRNA to eukaryotic cells. For the combined generation and delivery of eukaryote-translatable mRNA to eukaryotic cells, the system uses invasive, non-pathogenic bacteria to generate and deliver mRNA cargo to the eukaryotic cells. In the case of microbial production of eukaryote-translatable mRNA, the system uses non-pathogenic bacteria to generate mRNA that can be extracted in a form that is functional in eukaryotic cells. The bacteria contain at least one prokaryotic expression cassette encoding the mRNA on the chromosome or a plasmid; the mRNA contains a poly-A sequence transcribed by the bacteria and a 5′ cap or pseudo-cap element, e.g., an internal ribosome entry site (IRES) element, that mediates ribosome recruitment and translation in the eukaryotic host cell. Examples of therapeutic mRNA function include, but are not limited to, providing genetic material encoding antibodies and defective genes in the host. The promoter used in the present system driving expression of the mRNA within the bacterial cell is only operable within the bacteria and is not operable in a eukaryotic cell. The mRNA transcript that is generated and/or delivered with this system is translatable in the eukaryotic host cell at the time of extraction from the bacteria or at the time of bacterial-mediated delivery such that it can be translated into protein without additional post-transcriptional processing. This facilitates a more streamlined method of mRNA manufacturing and a shortened time to clinical effect if the mRNA is to be used for therapeutic applications. Examples of non-therapeutic mRNA function where the mRNA is used for general applications in research include, but are not limited to, providing genetic material for in vitro translation into a polypeptide.

In certain embodiments, the present invention provides mRNAs that can be utilized in therapeutic applications. However, this invention is agnostic to nature of mRNA is being generated and, in certain embodiments, delivered. It's not just therapeutic mRNA that we are making; mRNA is mRNA. For example, the mRNA generated and delivered might be for a therapeutic purpose, or it might be intended for in vitro research, such as to establish the effect of a particular mRNA in a cell or the effect of the polypeptide expressed from that mRNA.

In a first aspect the present invention provides a bacterial system to generate eukaryote-translatable mRNA. The system can include a bacterium having at least one prokaryotic expression cassette that requires a promoter only operable in a bacterial cell, wherein the prokaryotic expression cassette encodes at least one mRNA molecule, and wherein the mRNA molecule contains eukaryote-translatable elements for translation into a protein in a eukaryotic cell. The promoter used to drive expression of the prokaryotic expression cassette will generally be one that is not functional in a eukaryotic cell. The mRNA molecule can then be transcribed by a prokaryotic RNA polymerase. Within the context of the invention of the first aspect, the bacteria are engineered to express at least one mRNA molecule containing eukaryote-translatable elements from a sequence on the chromosome of the bacterium. The bacteria are transformed with at least one plasmid (also referred to as vector) designed to express at least one mRNA molecule containing eukaryote-translatable elements. The target eukaryotic cell can be an animal or plant cell, including a dividing or non-dividing cell. The mRNA molecule of the first aspect will have a 5′ cap or pseudo cap-like element capable of eukaryotic ribosome recruitment and a 3′ end containing a poly-A tail resulting in a eukaryote-translatable mRNA molecule produced within the bacterial cell. The eukaryote-translatable elements for translation into a protein include a viral or eukaryotic cellular internal ribosome entry site (IRES) element. In certain embodiments the encoded mRNA molecule has a bacterially transcribed poly-A region and a 5′ pseudo-cap element that will mediate translation initiation in the eukaryotic host cell via an internal ribosome entry site (IRES) element. It is further contemplated that the bacterium includes, such as via the plasmid, poly-A binding proteins for stabilization of a poly-A tail on the mRNA molecule. The poly-A region can contain 1-500 A's. In certain embodiments the bacterium is a Gram-negative or Gram-positive bacterium. The composition as defined in the first aspect can be used in medicine, for the prevention of disease, in therapy or in research applications. The composition as defined in the first aspect can be included in a pharmaceutically acceptable formulation.

In a second aspect the present invention provides a prokaryotic expression cassette. The prokaryotic expression cassette includes a prokaryotic promoter operable in a bacterial cell. The prokaryotic expression cassette encodes at least one mRNA molecule, where the mRNA molecule contains elements required for translation into a protein upon delivery to the cytoplasm of a eukaryotic cell. In certain embodiments the cassette further encodes a cell entry mediator and an endosomal release mediator. The cell entry mediator can be an invasin protein (e.g. as encoded by inv gene) or a fragment or binding domain thereof and the endosomal release mediator can be listeriolysin O (LLO) (e.g. hlyA gene). An IRES element, such as a viral IRES element, can be included in the sequence for the mRNA and in the 5′ region to the mRNA sequence to facilitate ribosomal recruitment. In certain embodiments the expression cassette has a 5′ end containing a cap or cap-like element capable of eukaryotic ribosome recruitment and a 3′ end containing a poly-A tail resulting in a eukaryote-translatable mRNA molecule produced within the bacterial cell. The poly-A region can contain 1 to about 500 A's. The prokaryotic expression cassette of the second aspect can be included in an invasive nonpathogenic bacterium.

The mRNA of various aspects can be a therapeutic mRNA having a function including, but not limited to, providing genetic material encoding antibodies or antibody fragments or providing genetic material rescuing defective genes in the host. The resultant transcribed mRNA molecule can be transcribed with elements to promote a circular conformation of the mRNA molecule.

In further aspects and embodiments, the mRNA molecule is produced in a biomanufacturing system and collected for downstream applications.

The eukaryote-translatable mRNA can be circularized in the bacteria upon its transcription. For example, a bacteriophage T4 permuted intron-exon (PIE) method can be used to promote circularization of the mRNA. Through group I intron self-splicing, splicing and then ligation of two exons occurs forming a circular RNA product which can theoretically be translated inside a eukaryotic cell. The circular eukaryote-translatable mRNA may in some instances be transcribed with a 3′ poly-A sequence. A circular eukaryote-translatable mRNA conformation may in some instances prove advantageous in that the 5′ and 3′ ends are inaccessible to RNases, thereby preventing degradation of the mRNA molecule and enhancing stability of the eukaryote-translatable mRNA. The present invention provides a bacterium that can transcribe either linearized eukaryote-translatable mRNA with a 5′ cap/pseudo cap and 3′ polyA tail or the bacteria can transcribe circular eukaryote-translatable mRNA. It is demonstrated experimentally herein that circular mRNA is made inside the bacteria with a viral IRES element on the 5′ end and with or without a polyA tail.

In a third aspect the present invention provides a system for generating eukaryote-translatable mRNA. The system of the third aspect can include a bacterium engineered to have at least one expression cassette encoding a eukaryote-translatable mRNA comprising a 5′ pseudo-cap element, a nucleic acid sequence encoding a polypeptide, and a poly-A tail, wherein transcription of the eukaryote-translatable mRNA is under the control of a prokaryotic promoter. The 5′ pseudo-cap element can be an internal ribosome entry sequence (IRES). In an advantageous embodiment the IRES is Cricket paralysis virus (CrPV) IRES, Foot and mouth disease virus (FMDV) IRES, Classical swine fever virus (CSFV) IRES or an IRES listed in tables 1-3. In further advantageous embodiments the bacterium is a nonpathogenic bacterium engineered to have at least one invasion factor.

The bacterium can be engineered to transcribe a eukaryote-translatable mRNA that is circularized in the bacteria upon its transcription.

In a fourth aspect the present invention provides a system for generating eukaryote-translatable mRNA. The system can include a nonpathogenic bacterium engineered to have at least one invasion factor and having at least one expression cassette encoding a eukaryote-translatable mRNA comprising an IRES, a nucleic acid sequence encoding a polypeptide, and a poly-A tail. Transcription of the eukaryote-translatable mRNA can be under the control of a prokaryotic promoter.

In a fifth aspect the present invention provides additional systems for generating eukaryote-translatable mRNA, The system of the fifth aspect can include a bacterium having at least one expression cassette comprising a sequence encoding a eukaryote-translatable mRNA, wherein transcription of the sequence encoding the eukaryote-translatable mRNA is under the control of a promoter that is inactive in a eukaryotic cell and wherein the eukaryote-translatable mRNA molecule comprises eukaryote-derived sequence elements that allow translation of a polypeptide in a eukaryotic cell.

The sequence encoding the eukaryote-translatable mRNA can be engineered to be on the chromosome of the bacterium. Alternatively, the expression cassette can be a plasmid comprising a sequence encoding at least one mRNA molecule containing eukaryote-translatable elements.

The expression cassette can have a sequence encoding a eukaryote-translatable mRNA that has a 5′-end comprising a 5′ cap or pseudo cap-like element capable of eukaryotic ribosome recruitment and a 3′ end containing a poly-A tail resulting in a eukaryote-translatable mRNA molecule produced within the bacterial cell. The eukaryote-translatable elements for translation into a protein can be a viral or eukaryotic cellular internal ribosome entry site (IRES) element. In an advantageous embodiment the viral or eukaryotic cellular internal ribosome entry site (IRES) element is selected from the group consisting of Cricket paralysis virus (CrPV) IRES, Foot and mouth disease virus (FMDV) IRES and Classical swine fever virus (CSFV) IRES.

The system for generating eukaryote-translatable mRNA according to claim 7 wherein the sequence encoding a eukaryote-translatable mRNA includes a sequence encoding poly-A region and a sequence encoding a 5′ pseudo-cap element capable of mediating translation initiation in the eukaryotic host cell via an internal ribosome entry site (IRES) element. The poly-A region can contain 1-500 A's.

In a sixth aspect the present invention provides additional systems for generating eukaryote-translatable mRNA comprising an engineered bacterium having a sequence encoding a eukaryote-translatable mRNA from the chromosome of the bacterium, wherein transcription of the eukaryote-translatable mRNA is under the control of a promoter that is inactive in a eukaryotic cell and the sequence encoding the eukaryote-translatable mRNA encodes a 5′ IRES and a 3′ poly-A tail. The promoter can be a prokaryotic promoter. The bacterium can be a non-pathogenic invasive bacterium. The nonpathogenic bacterium can be engineered to have at least one invasion factor to facilitate entry into the bacterium or release from a bacterial endosome.

In a seventh aspect the present invention provides system for generating eukaryote-translatable SARS-CoV-2 (or other coronavirus) mRNA encoding a spike protein comprising a bacterium having at least one expression cassette comprising a sequence encoding a 5′ IRES and a sequence encoding a eukaryote-translatable mRNA for a coronavirus spike polypeptide or fragment thereof, wherein transcription of the sequence encoding the eukaryote-translatable mRNA is under the control of a promoter that is inactive in a eukaryotic. The bacterium can be a non-pathogenic invasive bacterium. The nonpathogenic bacterium can be engineered to have at least one invasion factor to facilitate entry into the bacterium or release from a bacterial endosome. The invasion factor is encoded by an inv or hlyA gene. The promoter can be a prokaryotic promoter.

Some of the current vaccines for SARS-CoV-2 employ the administration of mRNA to the subject. Numerous short-comings exist in such vaccines, including the difficulty of producing large quantities of mRNA, the storage and handling of the vaccine compositions having the mRNA, and the delivery vehicle used to deliver the mRNA. The present invention provides a system that can be used to generate large quantities of mRNA. In addition, the system does not have the stringent handling requirements of current SARS-CoV-2 mRNA vaccine. Further, the production system can simultaneously serve as the delivery vehicle, simply delivery and reducing toxicity.

In a seventh aspect the present invention provides system for generating and delivering eukaryote-translatable viral antigen mRNA comprising a bacterium having at least one expression cassette comprising a sequence encoding a 5′ IRES and a eukaryote-translatable mRNA for viral polypeptide or fragment thereof, wherein transcription of the sequence encoding the eukaryote-translatable mRNA is under the control of a promoter that is inactive in a eukaryotic cell. The system for generating and delivering eukaryote-translatable viral antigen mRNA can be an antigen from a listed in Table 2.

In further aspects the present invention provides a method for treating or preventing disease in a subject. The method can include the step of administering a composition such as those described in the various aspects, above. The composition can delivered by intramuscular or intranasal administration, or by various routes as disclosed below.

The present invention further provides methods for making bacteria that can generate eukaryote-translatable mRNA such as disclosed in examples below. Briefly, nucleic acid sequences desired to be transcribed into eukaryote-translatable viral antigen mRNA can be cloned into expression cassettes encoding pseudo-cap elements and poly-A tails. In advantageous embodiments the bacteria can be nonpathogenic bacteria engineered to express one or more invasion factors.

### BRIEF DESCRIPTION OF THE DRAWINGS

For a fuller understanding of the invention, reference should be made to the following detailed description, taken in connection with the accompanying drawings, in which:

FIG. 1 is a drawing showing mRNA with a bacterially transcribed 5′ element capable of recruiting eukaryotic ribosomes to the RNA (in this example, an IRES element) and 3′ poly-A sequence.

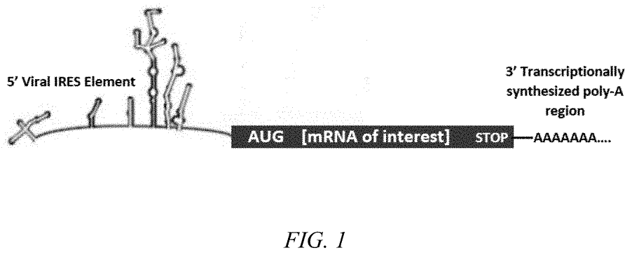

FIG. 2 is a drawing showing the plasmid design for an mRNA production and delivery system; the embodiment is depicted with a viral IRES element, which functions like a 5′ cap, and with a 3′ poly-A sequence.

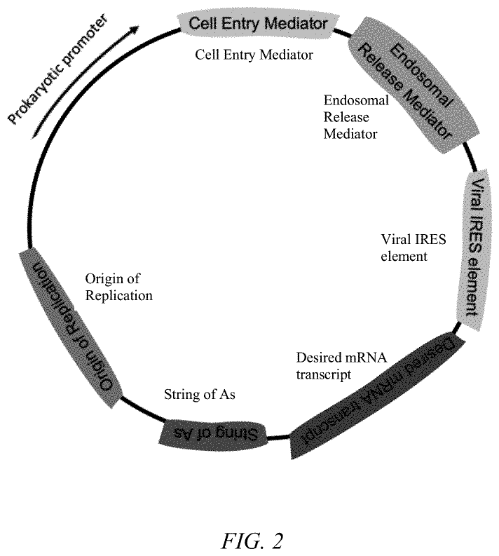

FIG. 3 is a drawing depicting a possible therapeutic application of the invention where the bacterial system is used to generate and deliver eukaryote-translatable mRNA via inhalation or aerosolized delivery to a mouse.

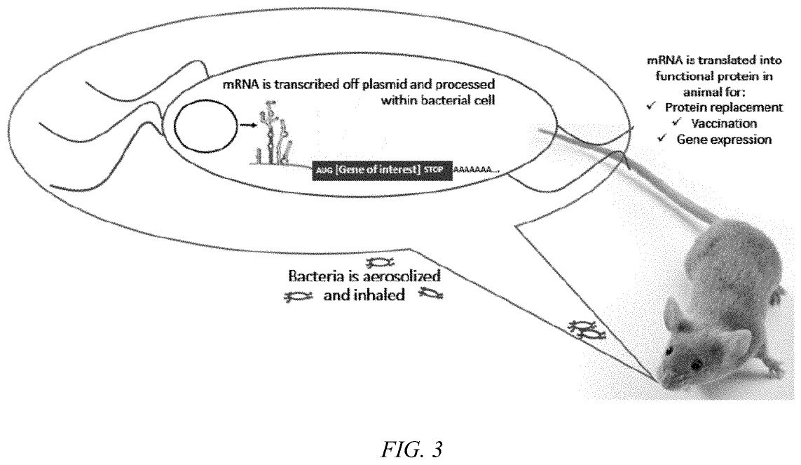

FIG. 4 is an image of an agarose gel with PCR products verifying the presence of the CrPV IRES element (104 bp) and the mammCh gene (699 bp) coding sequence in bacterially transcribed eukaryote-translatable mRNA.

FIG. 5 is a set of three images showing bacteria transformed to express RFPs with prokaryotic or eukaryotic RBSs, imaged in the RFP channel on the Nexcelom Celigo. FIG. 5 demonstrates that while bacteria alone show red fluorescence when they express E2-Crimson in the presence of a prokaryotic RBS, they do not show red fluorescence when the mammCh sequence is downstream from the CrPV eukaryotic IRES sequence or when carrying the scramble plasmid as a negative control.

FIG. 6 is a set of six images showing A549 lung epithelial cells incubated with bacteria expressing eukaryote-translatable mRNA, imaged in the brightfield and RFP channels on the Nexcelom Celigo. FIG. 6 demonstrates that while A549 cells treated with bacteria expressing a scramble negative control sequence do not show red fluorescence, A549 cells treated with bacteria expressing the mammCh sequence downstream from a CrPV eukaryotic IRES sequence show robust red fluorescence signal.

### DETAILED DESCRIPTION OF THE PREFERRED EMBODIMENT

This invention relates to a prokaryotic expression system for the production of eukaryote-translatable mRNAs within a bacterial cell, in which the eukaryote-translatable mRNAs accumulate inside the bacterium, and the eukaryote-translatable mRNAs are collected from the bacterial cells or remain in the bacterial cells for subsequent delivery to eukaryotic cells so that the eukaryote-translatable mRNAs and are capable of being translated into a protein inside of a eukaryotic cell. This invention additionally relates to the treatment and prevention of disease. More specifically, this invention relates to a prokaryotic expression system to generate mRNAs within a bacterial delivery vehicle for delivery to a eukaryotic host cell and immediate translation into a protein.

The present invention provides an mRNA production and delivery system that, in certain embodiments, can employ an invasive, non-pathogenic bacterial cell to generate, and in some instances also to deliver, the mRNA for translation in eukaryotic cells. The bacterial cell can contain a prokaryotic expression cassette encoding the mRNA and mechanisms or sequences for capping, or pseudo-capping, and polyadenylating the mRNA within the bacterial cell. The translatable mRNA can be encoded from a plasmid or from sequences on the chromosome of the bacterium, still under the control of a prokaryotic promoter. Where delivery to the eukaryotic cell is not desired, the system can employ non-invasive or invasive bacterial cells to generate the mRNA, such as an mRNA with a pseudo-cap and poly-A sequence.

In vitro generation of non-translatable RNA has been established, wherein non-translatable RNAs are transcribed either in bacteria, or synthesized using chemical approaches or in a test tube with the required components and enzymes. This form of RNA does not contain the 5′ and 3′ elements required for eukaryotic translation, which include a 7-methylguanosine nucleotide at the 5′ end, herein referred to as a “5′ cap,” and a sequence containing only adenine bases at the 3′ end, herein referred to as a “poly-A tail.” The RNA must therefore be further processed into mRNA by exogenous capping and tailing with enzymes, or the DNA encoding this RNA sequence must be integrated into a eukaryotic host genome, transcribed by the eukaryotic cell, and endogenously capped and tailed using the host cell's natural capping and tailing mechanisms. The 5′ cap and a 3′ poly-A tail are required for mRNA stability, ribosome recruitment, and translation of the mRNA into protein. The 5′ cap structure mediates ribosomal association, physically bringing together the necessary cellular machinery and components that translate the mRNA transcript into a protein. The poly-A tail protects the mRNA from enzymatic degradation in the cytoplasm and aids in transcription termination, export of the mRNA from the nucleus, and is necessary for translation into a protein. The 5′ cap and the 3′ poly-A tail both protect the mRNA from degradation by RNases prior to translation, improving transcript stability in the cell. In vitro generation of functional, translatable (fully processed, capped and tailed) mRNAs is limited by the complexity of the multi-step process involved to generate and then separately process the mRNA to contain a 5′ cap and a 3′ poly-A tail. In vivo processing of mRNA by a eukaryotic cell to add the 5′ cap and 3′ poly-A tail, rendering it functional and translatable, is also constrained by the potential for off-target and deleterious effects when the mRNA is integrated into the host genome. The current multistep procedures are highly limiting for downstream commercialization and manufacturing, as well as general application in research or clinical settings.

Historically, mRNAs are made synthetically and modified chemically so as to contain the necessary elements for translation into proteins. These synthetic mRNAs are typically delivered in one of three general ways: via liposomes, nanoparticles, or as a conjugate. However, these delivery methods have profound limitations, including immunogenic effects, short half-life, elevated toxicity (compared to naked mRNA), etc. [Kaczmarek et al. “Advances in the delivery of RNA therapeutics: from concept to clinical reality,” Genome Med. 9:1-16 (2017)]. Another challenge associated with these delivery methods is the inability to deliver large, negatively charged mRNA molecules into the target eukaryotic cell due to constraints associated with crossing the cell membrane. Additionally, current methods for mRNA delivery fail to enable targeting of specific tissues, cell types, and locations within the body. This deficiency means that systemic administration is required, which can compound problems with toxicity and immunogenicity, in addition to increasing the cost of treatment as a result of requiring more mRNA to achieve the same dose as would be required if delivered specifically to a targeted tissue or body location. Some mRNAs have been delivered via a viral vector [Zhong et al. “mRNA therapeutics deliver a hopeful message,” Nanotoday 23:16-39 (2018)]. Viral vectors also have problems with immunogenicity and insertional mutagenesis, and are difficult to produce under GMP conditions, which is important for human clinical use. Viral vectors can also be immunogenic and stimulate a deleterious antibody response in a patient.

Despite limitations to delivery, several mRNA therapeutics are currently available for human use. One example is Gendicine®, a viral vector/delivery vehicle that encodes the p53 tumor protein used for treatment of head and neck cancer. A second example is Glybera®, a viral vector/delivery vehicle that encodes lipoprotein lipase and is used for protein replacement in patients who are deficient in lipoprotein lipase. Both viral vectors rely on eukaryotic transcription of the mRNA therapeutic from a viral vector-delivered DNA template containing eukaryotic gene regulatory elements, meaning the viral vector is not capable of delivering pre-made eukaryote-translatable mRNA to a host cell. Note that the term “vector”, as occasionally used in the literature, can sometimes refer to a delivery vehicle, such as a liposome, viral vector, or bacterial delivery vehicle. As generally used herein, the bacterium of the present invention may contain the expression unit for the eukaryote-translatable mRNA within a vector autonomously replicable separately from the chromosome, such as a plasmid, cosmid, bacterial artificial chromosome, bacteriophage, or any extrachromosomal element, which would correspond to a more traditional view of the term “vector”.

Although there have been great advances in the field, and targets for RNA therapeutics are replete, a comprehensive self-contained system for robust mRNA generation and non-immunogenic, non-toxic, efficient delivery has yet to be established. While mRNA has great potential for applications such as protein replacement and vaccination, the lack of a delivery mechanism that is non-immunogenic, tissue specific, non-integrative, and capable of generating a translatable mRNA molecule using its own gene expression functions significantly limits the field. Although mRNA has demonstrated efficacy when used as a therapeutic, an improved means of delivery must be established to bring additional mRNA drugs to the clinic. The current state-of-the-art lacks the capacity to function as a complete system for mRNA generation, including the production of mRNA species with a 5′ cap or pseudo-cap element and a 3′ poly-A tail such that they are competent for eukaryotic translation before delivery and can be translated into protein by the eukaryotic host cell immediately upon delivery, or otherwise when the generated mRNA is used for research or therapeutic applications. Additionally, the state-of-the-art for mRNA therapeutics poses safety risks, as current approaches (e.g. viral vectors) require integration into the host's genome for mRNA processing (transcription) prior to translation, often resulting in adverse immune-related effects and potentially deleterious genome destabilization.

The present invention provides a novel bacterial system for the production or biomanufacture of 5′-capped and 3′-polyadenylated mRNA that is translatable when delivered to a eukaryotic cell. In some instances, this bacterial system can additionally provide targeted delivery to specific cells and tissues via ligand-specific receptor targeting. The system also provides a mechanism for intracellular uptake of mRNA molecules by any eukaryotic cell (dividing and non-dividing) via receptor-mediated endocytosis, without eukaryotic-cell genomic integration, thereby abating potential complications, including tumorigenesis caused by insertional mutagenesis upon integration into the host genome. Building upon a bacterial platform for delivery of nucleic acids in general, this invention encompasses a novel means of eukaryote-translatable mRNA generation that occurs entirely within a bacterial cell under the control of a prokaryotic promoter, such that the eukaryote-translatable mRNAs transcribed within the bacterial cells are transcribed with the required 5′ and 3′ elements and thereby translatable prior to delivery to a eukaryotic cell.

There are numerous advantages to this system of a bacterially mediated mRNA production and delivery system compared to other mRNA production and delivery methods. The details and some of the more significant advantages are discussed below.

Self-contained system: The bacterial cells are multi-functional for both generation of eukaryote-translatable mRNA and, if desired, delivery of the eukaryote-translatable mRNA to eukaryotic cells. These bacterial cells serve as the site of eukaryote-translatable mRNA production and can also serve as the delivery vehicle for fully translatable mRNA to specific eukaryotic host cells and tissues. Production of a desired eukaryote-translatable mRNA can be achieved by transforming the bacterial cells, such as with a plasmid encoding the mRNA of interest under the control of a prokaryotic promoter as described herein. The transformed bacteria can also function as the delivery vehicle where the bacteria are a naturally invasive strain of bacteria or a strain of bacteria that has been engineered to be invasive, such as by inclusion of an invasion factor on a plasmid or on the chromosome of the bacteria. The bacterial cells are capable of efficient replication in media for scalable biomanufacturing. This is in contrast to other mRNA delivery systems that require complicated and expensive multi-step manufacturing approaches. Bacterial strains encoding different eukaryote-translatable mRNAs can also be easily frozen in glycerol where they remain viable for later retrieval as needed.

Rapidly effective: The novel bacterial delivery system of the present invention achieves the desired eukaryote-translatable mRNA delivery event rapidly without eukaryotic host genome integration or further mRNA processing, thereby supporting rapid translation into a protein and eliminating non-specific effects in the eukaryotic host cell to which it was delivered. Since the eukaryote-translatable mRNA is delivered in a fully functional form, there is no need for processing in the eukaryotic cell, and the time to clinical effect is shortened as the cell may immediately translate the delivered eukaryote-translatable mRNA.

Non-immunogenic: The bacterial delivery vehicle evades antigen presenting cell recognition due to a lipopolysaccharide-rough phenotype, and in vivo data indicate that the system does not induce innate or adaptive immune responses or any other cytokine cascades in the host. In its non-immunogenic nature, the present vehicle starkly differs from other delivery vehicles, including nanoparticles, liposomes, and viral vectors, which can stimulate innate and adaptive immune responses, potentially leading to antibody production.

Non-integrative: Transcription of the complete mRNA molecule is exclusively controlled by prokaryotic promoters. This means that the mRNA is fully transcribed as a eukaryote-translatable mRNA by the bacterial cell. This feature prevents the need for DNA integration into the eukaryotic host genome, provides controlled delivery of the mRNA product, and eliminates risk of unwanted side-effects due to aberrant host genome integration.

Highly stable: The delivery system is not inhibited by exposure to serum, proteases, or nucleases, allowing the bacterial vehicles and eukaryote-translatable mRNA cargo to remain stable until they reach their target site. Unlike other non-viral vectors, the bacterial delivery vehicles are not eliminated by phagocytic clearance, which additionally contributes to stability. Naked mRNA has a short half-life and is susceptible to degradation by nucleases. The system of the present invention is more robust than delivery of naked mRNAs due to the secure environment provided by the bacterial cell until the eukaryote-translatable mRNA cargo has reached its destination inside the target eukaryotic cell. The bacterial delivery vehicles shield the eukaryote-translatable mRNA from degradation before it reaches its target eukaryotic cell. The presence of a 5′ cap or pseudo-cap and 3′ poly-A tail prior to delivery further stabilizes the eukaryote-translatable mRNA transcript after delivery, increasing the probability of rapid translation into protein within the eukaryotic host cell.

Large delivery capacity: The bacterial system of the present invention can effectively generate and deliver large quantities of eukaryote-translatable mRNA, (e.g. >100:1; mRNA molecules per bacterial cell) as compared to a lipid nanoparticle (1:1; mRNA molecule per lipid nanoparticle) as well as multiple different mRNAs if desired. For example, a cocktail/population of bacteria can be created comprising a plurality of subpopulations of bacteria, where each subpopulation encodes a different eukaryote-translatable mRNA. It is further contemplated that a bacterium could be engineered to produce more than one eukaryote-translatable mRNA via the inclusion of more than one prokaryotic expression cassette. Moreover, those mRNAs could be under the control of different promoters, with promoters selected based upon strength to tailor relative eukaryote-translatable mRNA production levels within the bacteria. For example, a strong prokaryotic promoter could be used to produce a eukaryote-translatable mRNA where a high concentration is desired, while a weaker promoter could be used to control transcription of a eukaryote-translatable mRNA where a reduced amount of the transcript is desired. Concentrations of mRNA can be modulated based on plasmid copy number, chromosomal position, prokaryotic promoter strength, and time allotted for bacterial growth. This system uses receptor-mediated endocytosis for effective intracellularization of the bacterial vehicles and facilitates endosomal perforation and release of the mRNA into the eukaryotic cytoplasm, i.e., the site of protein translation.

Cost effective production: This bacterial system represents a bio-production (biomanufacturing) system for eukaryote-translatable mRNA that provides a more cost-effective method of manufacturing compared to both conventional enzymatic synthesis of mRNA and other bioproduction systems that do not produce fully processed (5′ capped and 3′ poly-A tailed) mRNA molecules. This mode of bacterial mRNA production represents an efficient one-step method for producing eukaryote translatable mRNA in contrast to other systems that require at least three steps to produce the synthetic RNA, add a synthetic 5′ cap, and enzymatically polyadenylate similar mRNA products. For this reason, using the bacterial system of the present invention for biomanufacturing of eukaryote-translatable mRNA offers a more advanced, efficient, and cost-effective means of producing eukaryote-translatable mRNA by requiring less time, less resources (reagents, instrumentation, manpower), permitting production of multiple eukaryote-translatable mRNA sequences simultaneously, and allowing for larger-scale production of eukaryote-translatable mRNA.

The present invention advances the state of the art for numerous reasons, many of which are discussed below.

First, the present invention provides a system that can accomplish both the expression and delivery of eukaryote-translatable mRNA in a self-contained system, in contrast to merely enabling the generation of RNA, which would then require a second independent step to add a 5′ cap and 3′ poly-A tail to the RNA molecules, such as in the target cell or in a test tube following isolation of bacterially generated RNA.

Second, the system of the present invention uses a prokaryotic expression cassette that is only operable using prokaryotic promoters and, accordingly, bacterial polymerases, to generate fully functional mRNAs (5′-capped and 3′ polyadenylated mRNAs) that are ready to be translated in and by the eukaryotic host cells immediately upon delivery to the cytoplasm. Production of the eukaryote-translatable mRNAs occurs within the bacterial delivery vehicle (the bacterial cell), thereby simplifying and streamlining the process of synthesis and delivery. Importantly, because the present system uses a prokaryotic expression cassette to drive mRNA expression, it also mitigates risk of aberrant integration into the eukaryotic host cell genome. This is in contrast to a system that uses bacteria to deliver eukaryotic expression cassettes that express mRNA using eukaryotic promoters that are only recognized by eukaryotic polymerases, so that upon delivery to host cells, the delivered expression cassette integrates into the host genome and the mRNA is transcribed by the eukaryotic cell and subsequently translated into protein. The system of the invention accomplishes mRNA transcription and processing into a translatable mRNA, all while inside the bacterial cell. This is one feature that contributes to the transitory nature of mRNA production with the present system, which can be of great value in situations where providing a finite quantity of mRNA is desirable (i.e. to reduce off-target effects to the patient) and where long-term production of the mRNA might not be necessary or desirable.

Third, the present invention generates and can deliver eukaryote-translatable mRNA molecules for translation into polypeptides in a eukaryotic host cell using the host cell translation system. This differs from a system that delivers pre-made proteins or polypeptides (e.g., antigens, enzymes, antibodies) directly to the eukaryotic host cell. The present invention can generate and deliver more eukaryote-translatable mRNA molecules that can guide the production of a higher protein concentration than could be delivered if already in protein form. Additionally, delivering eukaryote-translatable mRNA to the eukaryotic host cell allows the host cell to generate the protein, further ensuring that the protein is properly folded (necessary for protein function), whereas delivering protein to a eukaryotic cell requires that the protein being delivered is already properly folded prior to delivery to the eukaryotic cell. Eukaryotic post-translational processing mechanisms, which facilitate functions such as protein folding, methylation, and phosphorylation, are often different from prokaryotic mechanisms and can be difficult to replicate in a test tube.

The present invention is significantly safer compared to other technologies due to the non-integrative and non-immunogenic nature of the bacterial cell that generates and delivers the eukaryote-translatable mRNA. These features further reduce the potential for toxicity. The tissue-specific delivery afforded by the present system, whereby the bacteria express invasion factors that facilitate bacterial uptake via receptor mediated endocytosis into specific cells associated with specific tissue types, e.g., eye, reproductive organs, lungs, muscle, and other epithelia, is superior to systems that deliver mRNA via systemic administration. This self-contained bacterial delivery system produces the desired eukaryote-translatable mRNA containing an element functionally equivalent to a canonical eukaryotic 5′ cap (referred to herein as a “pseudo-cap”) and a 3′ poly-A tail within the bacterial cell, and subsequently the bacteria can deliver this fully functional mRNA intracellularly into specific tissues within a eukaryotic host organism. Therefore, this system is not only relevant for in vitro applications, but also for in vivo applications, where the eukaryote-translatable mRNA can be immediately processed into a polypeptide that can induce an intended therapeutic effect. Packaging of the processed eukaryote-translatable mRNAs within the bacterial delivery vehicle also provides protection to the eukaryote-translatable mRNAs during administration, thus modulating the concentration requirements to efficiently maximize the therapeutic effect of the eukaryote-translatable mRNA.

### Example 1: Generation of Invasive Bacteria Expressing Eukaryote-Translatable mRNA Encoding mCherry Fluorescent Protein

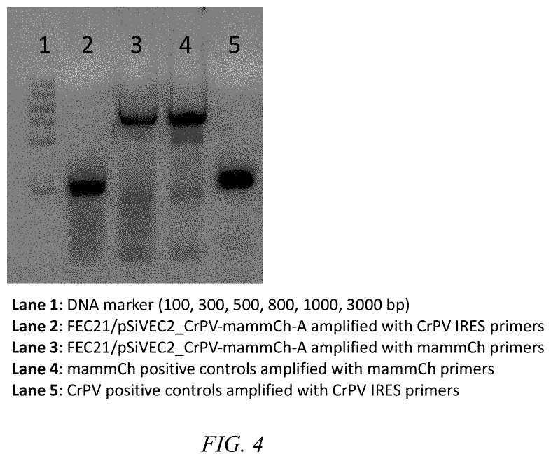

The pSiVEC2_CrPV-mammCh-A plasmid was constructed by cloning an internal ribosome entry site (IRES) element from cricket paralysis virus (CrPV) into the pSiVEC2 plasmid upstream of a mammalian codon-optimized mCherry (mammCh) coding sequence fused to a sequence of approximately 60 adenosine (A) residues, which together comprise a poly-A tail. The resulting plasmid encodes an RNA molecule comprising an IRES element, mammCh coding sequence, and a polyA tail, which together comprise a functional eukaryotic mRNA molecule to be transcribed as a eukaryote-translatable mRNA, which is expected to be translatable by a eukaryotic cell. pSiVEC2_CrPV-mammCh-A was transformed into E. coli bacteria (FEC21) to generate the strain FEC21/pSiVEC2_CrPV-mammCh-A. The FEC21 bacteria were additionally engineered to be invasive to eukaryotic cells via integration of the inv and hlyA genes for invasin- and receptor-mediated endocytosis (RME) and LLO-mediated endosomal release, respectively. FEC21 cells transformed with pSiVEC2_CrPV-mammCh-A were plated onto brain heart infusion (BHI) agar containing appropriate antibiotics for selection. Resulting colonies were screened via PCR to confirm the presence of pSiVEC2_CrPV-mammCh-A, amplifying the CrPV IRES element (104-base pair (bp) PCR product) and the mammCh-encoding sequence (699-bp PCR product) (FIG. 4). A single clone of FEC21/pSiVEC2_CrPV-mammCh-A was frozen at −80° C. in 20% glycerol. A single frozen aliquot from the stock was thawed for plate enumeration. Briefly, a 1-mL aliquot was centrifuged for 5 min at 5000×g and the cells were resuspended in 1 mL of BHI. The resulting bacterial suspensions were serially diluted and plated in triplicate on BHI agar containing antibiotics. Colony counts at each dilution were averaged to calculate the overall colony forming units (CFU)/mL and represented a viable concentration for stocks of FEC21/pSiVEC2_CrPV-mammCh-A. This system allowed a quantitated, live inoculum stock to be directly used in all future assays.

A standard invasion assay was used to test for eukaryotic translation of the bacterially expressed mRNA. Human alveolar basal epithelial cells (A549) were maintained in Dulbecco's modified Eagle's Medium (DMEM) supplemented with 10% fetal bovine serum, 2 mM GlutaMAX, 100 U/mL penicillin, and 100 g/mL streptomycin at 37° C. with 5% CO₂ incubation. Invasive bacteria (encoding inv and hlyA) can enter mammalian cells, including but not limited to A549 cells, via RME, thereby delivering their bacterially encoded and expressed cargo. The invasion assay includes the following steps.

A549 cells were seeded at fixed concentration into black-walled 24-well plates. On the day of bacterial invasion, three bacterial stocks were thawed: 1) FEC19/pE2Crimson (a non-invasive positive-control strain carrying a plasmid encoding the E2-Crimson fluorescent protein with a bacterial ribosome-binding site); 2) FEC21/pSiVEC2 Scramble (an invasive negative control strain carrying a plasmid encoding non-translatable and non-encoding scramble sequence); 3) FEC21/pSiVEC2_CrPV-mammCh-A (an invasive strain carrying a plasmid encoding the poly-adenylated mammCh mRNA under the control of the eukaryotic CrPV IRES ribosome-binding site). The bacteria were then prepared for the invasion assay as follows: Enumerated stocks frozen in glycerol were thawed from −80° C. and centrifuged for 5 min at 5000×g. The bacterial pellets were resuspended in DMEM(−) (serum- and antibiotic-free, high-glucose DMEM). at a final concentration of 2.5×10⁷ CFU/mL. FEC21/pSiVEC2_CrPV-mammCh-A cells were resuspended at two additional final concentrations of 1.25×10⁷ CFU/mL and 5×10⁷ CFU/mL. A549 cells were washed in DMEM(−) to remove antibiotics, and incubated for 2 hours (37° C. with 5% CO₂) with 0.5 mL of each bacterial suspension and subsequently rinsed 5× with DMEM(−) to remove unbound bacterial cells. Twenty-four hours after this treatment, cells were imaged using a Nexcelom Celigo instrument in the RFP channel (excitation 531/emission 629) to detect red fluorescence, representing E2Crimson or mammCh for FEC19/pE2Crimson and FEC21/pSiVEC2_CrPV-mammCh-A, respectively, and in brightfield, to observe cell density.

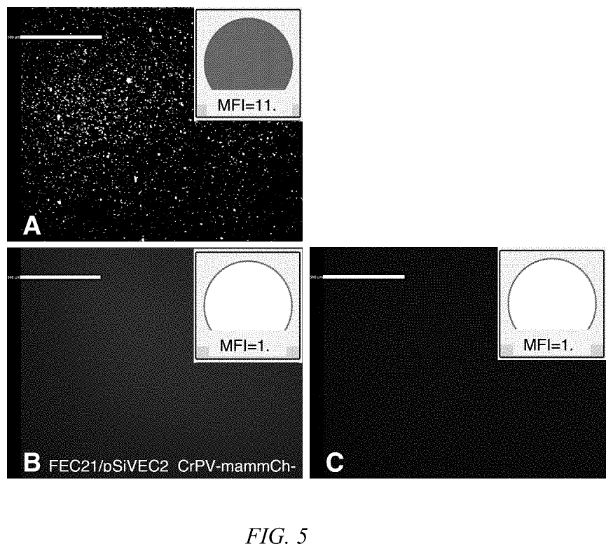

FIG. 5 demonstrates that while bacteria alone show red fluorescence when they express E2-Crimson in the presence of a prokaryotic RBS, i.e., FEC19/pE2Crimson (A), they do not show red fluorescence when the mammCh sequence is downstream from the CrPV eukaryotic IRES sequence (i.e., FEC21/pSiVEC2_CrPV-mammCh-A). (B). The bacteria carrying the scramble plasmid (pSiVEC2 Scramble) show no red fluorescence (C). In all panels, the scale bar represents 500 μm. The top right corner of each panel also depicts the mean fluorescent intensity of the sample well measured in the RFP channel on the Nexcelom Celigo instrument, which confirms the presence of an RFP signal from the E2-Crimson fluorophore and the absence of detectable RFP from the scramble and mammCh.

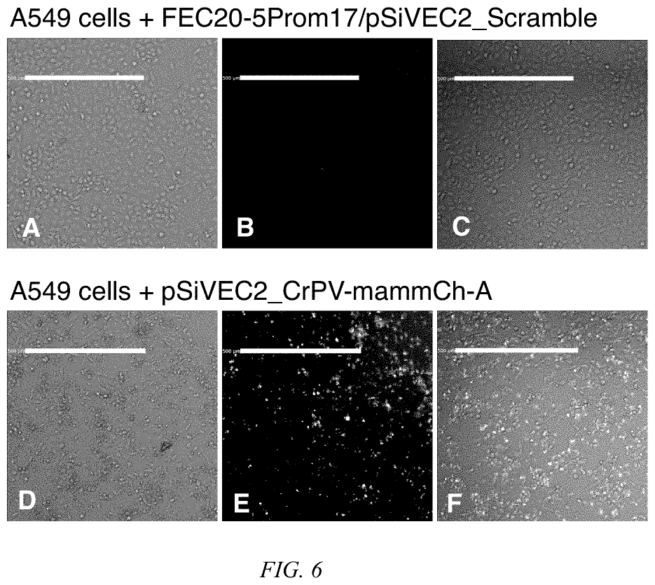

FIG. 6 demonstrates that while A549 cells treated with FEC21/pSiVEC2 Scramble do not show red fluorescence [brightfield in (A), red fluorescence channel (B), merge (C)], A549 cells treated with FEC21/pSiVEC2_CrPV-mammCh-A show robust red fluorescence signal [brightfield in (D), red fluorescence channel (E), merge, confirming colocalization of the red fluorescent signal and the A549 cells (F)]. In all panels, the scale bar represents 500 μm.

Together, these results demonstrate delivery and subsequent eukaryotic translation of a bacterially expressed mRNA molecule (eukaryote-translatable mRNA) by a bacterial cell.

### Example 2: Bacterial Transcription of Eukaryote-Translatable mRNA Molecules and Delivery to Mammalian Cells

Successful transcription of mRNA containing a 5′-IRES element, a gene coding sequence, and a 3′-polyA tail was demonstrated using a standard invasion assay and molecular detection techniques.

Four plasmid variants (Table 4) were constructed by cloning an IRES element into the pSiVEC2 plasmid upstream of a wildtype firefly luciferase (luc) coding sequence fused to a sequence of approximately 60 adenosine (A) residues, which together comprise a poly-A tail. The resulting plasmids encode an RNA molecule comprising an IRES element, luc coding sequence, and a poly-A tail, which together comprise a functional eukaryotic mRNA molecule, expected to be translatable by a eukaryotic cell. Each of the four plasmids was separately transformed into E. coli bacteria (FEC21) which were engineered to be invasive to eukaryotic cells via integration of the inv and hlyA genes for invasin- and receptor-mediated endocytosis (RME) and LLO-mediated endosomal release, respectively. Transformed FEC21 were plated onto BHI agar containing appropriate antibiotics for selection. Resulting colonies were screened via PCR to confirm the presence of both the IRES element (product sizes listed in Table 4) and the luc gene (513 bp product). The “pSIVEC2 circCrPV-lucA” construct was screened by an additional PCR to confirm the circular confirmation using a primer which spans the splice junction for ribozyme directed splice site for ribozyme directed mRNA circularization and would only be expected to produce an amplicon for circular and not linear mRNA; all such constructs evaluated tested positive for the 216 bp PCR product. Cultures were prepared from each of two isolated colony of each strain and grown to late log phase (OD₆₀₀ 0.8-1.0) with incubation at 37° C. in BHI medium with appropriate antibiotics.

RNA was extracted from the bacteria listed in Table 5 to demonstrate successful transcription of the eukaryote-translatable mRNA species within the bacterial cells. Briefly, approximately 5×10⁸ CFU of each bacterial culture was homogenized using 1 mm zirconia beads and a BioSpec BeadBeater. Total RNA was extracted using the Qiagen RNeasy Mini Kit, following the manufacturer's recommended protocol. The resulting RNA extracts were frozen at −80° C. until reverse transcription and PCR described in subsequent steps.

A standard invasion assay was also used to demonstrate bacterial delivery of the eukaryote-translatable mRNA transcripts to mammalian cells. Human A549 cells were cultivated as described in Example 1 and seeded at a fixed concentration in 6-well plates. The same bacterial cultures which were used in the above RNA extractions were prepared to approximately 2.5×10⁷ CFU/mL and 1 mL was incubated with A549 cells for 2 hours (37° C. with 5% CO2) and subsequently rinsed 5× with DMEM(−) to remove unbound bacterial cells. Complete DMEM, supplemented as detailed in Example 1, including with 100 U/mL penicillin, and 100 g/mL streptomycin was added to kill any remaining extracellular bacteria and incubated for another 2 hours. The A549 cells were washed another 3× with DMEM(−) and then detached with 750 μL TrypLE Express enzyme. RNA extractions were performed as described above using the entire cell volume.

All RNA sample concentrations and purity were measured by NanoDrop spectrometry and 1 ug of RNA was used in duplicate reverse transcription (RT) reactions using Promega AMV Reverse Transcriptase and primed with random hexamer or oligo(dT) primers. Random hexamer primers would be expected to enable RT of all bacterial and eukaryotic RNA transcripts. Oligo(dT) primers require the presence of a poly-A sequence which is absent from prokaryotic RNA so they would be expected to only enable RT of bacterial transcripts containing a poly-A tail, or canonical eukaryotic mRNAs as the case may be for endogenous mRNAs produced by the A549 cells.

Following RT, a fixed mass of the resulting cDNA was amplified by PCR and then electrophoresed on a 2% agarose gel to detect each of the necessary elements of the eukaryote-translatable mRNA. The PCR results summarized in Table 5 confirm that all of the components were present, including each of the different IRES elements evaluated and the gene coding sequence (luc), and the presence of the poly-A tail was verified by RT with oligo(dT) primers.

In summary, the results demonstrate 1) transcription of a circular eukaryote-translatable mRNA conformation inside a bacterial cell with the design described in the present invention, 2) successful bacterial transcription of RNA containing a 5′ IRES element acting as a pseudo-cap and a 3′ poly-A sequence which comprise elements required for eukaryotic translation, and 3) successful delivery of the bacterially generated eukaryote-translatable mRNA (both linear and circular) to eukaryotic cells in detectable quantities.

### Glossary of Claim Terms

As used throughout the entire application, the terms “a” and “an” are used in the sense that they mean “at least one”, “at least a first”, “one or more” or “a plurality” of the referenced components or steps, unless the context clearly dictates otherwise. For example, the term “a cell” includes a plurality of cells, including mixtures thereof.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements. Furthermore, when numerical ranges of varying scope are set forth herein, it is contemplated that any combination of these values inclusive of the recited values may be used.

As used herein, and particularly in the claims, the term “comprising” is intended to mean that the products, compositions and methods include the referenced components or steps, but do not exclude others. “Consisting essentially of” when used to define products, compositions and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

Kits for practicing the methods of the invention are further provided. By “kit” is intended any manufacture (e.g., a package or a container) comprising at least one reagent, e.g., a pH buffer of the invention. The kit may be promoted, distributed, or sold as a unit for performing the methods of the present invention. Additionally, the kits may contain a package insert describing the kit and methods for its use. Any or all of the kit reagents may be provided within containers that protect them from the external environment, such as in sealed containers or pouches.

In an advantageous embodiment, the kit containers may further include a pharmaceutically acceptable carrier. The kit may further include a sterile diluent, which is preferably stored in a separate additional container. In another embodiment, the kit further comprising a package insert comprising printed instructions directing the use of a combined treatment of a pH buffer and the anti-pathogen agent as a method for treating and/or preventing disease in a subject. The kit may also comprise additional containers comprising additional anti-pathogen agents (e.g., amantadine, rimantadine and oseltamivir), agents that enhance the effect of such agents, or other compounds that improve the efficacy or tolerability of the treatment.

In the present invention, the term “bacterium having a eukaryote-translatable mRNA-producing ability” refers to a bacterium having an ability to express and accumulate the eukaryote-translatable mRNA in cells of the bacterium to such a degree that the eukaryote-translatable mRNA can be collected when the bacterium is cultured in a medium. The bacterium having the eukaryote-translatable mRNA-producing ability may be a bacterium that can accumulate the heterologous, eukaryote-translatable mRNA in the bacterial cells in a quantifiable amount. In one embodiment, the bacterial strain may be modified so that the activity of ribonuclease III (RNase III), other ribonucleases (RNases), or other enzymes that can degrade of modify RNA, e.g., PNPase, is reduced or eliminated. The bacterium having the eukaryote-translatable mRNA-producing ability may also be a bacterium that can accumulate the eukaryote-translatable mRNA in the bacterial cells in an amount of 1 picogram/L or more, 1 mg/L-culture or more, 2 mg/L-culture or more, 5 mg/L-culture or more, 10 mg/L-culture or more, 20 mg/L-culture or more, 50 mg/L-culture or more, or 100 mg/L-culture or more.

As used herein, the term “bacterium” or “bacteria” is intended to mean any Gram-positive or Gram-negative bacterium. In one embodiment, a coryneform bacterium can be used as the eukaryote-translatable mRNA-producing strain. Examples of the coryneform bacterium include bacteria belonging to the genera Corynebacterium, Brevibacterium, Mycobacterium, Microbacterium, or the like. In some instances, the Corynebacterium is Corynebacterium glutamicum. Additionally, the bacterium used for production of eukaryote-translatable mRNA may be generally regarded as safe (GRAS) microorganisms.

In one embodiment of the present invention, one or more nucleic acid sequences (e.g., a DNA sequence or an RNA molecule), each corresponding to a eukaryote-translatable mRNA, may be produced by a bacterium.

The eukaryote-translatable mRNA sequence is not limited so long as it is exogenous RNA and/or RNA other than the RNA naturally found in the bacterial strain producing the eukaryote-translatable mRNA. Alternatively, or in addition, the RNA will be transcribed to contain a 5′-cap or 5′-pseudo cap and a 3′-poly-A tail. Thus, the eukaryote-translatable mRNA will not be an RNA naturally found within the bacterial strain producing eukaryote-translatable mRNA, but instead will be a product of man. The eukaryote-translatable mRNA can be appropriately selected for production inside the bacterium according to various conditions, applications, and purposes of use of the eukaryote-translatable mRNA. The eukaryote-translatable mRNA may be, for example, RNA as it would exist naturally without modification (but made unnaturally, existing such as through cloning into a plasmid and/or bacterium), modified RNA thereof, or artificially designed RNA. The eukaryote-translatable mRNA may be, for example, RNA derived from a virus, RNA derived from a microorganism, RNA derived from an animal, RNA derived from a plant, or RNA derived from a fungus. The eukaryote-translatable mRNA may be, for example, RNA that encodes for a protein antigen associated with a coronavirus, such as the SARS-CoV-2 virus strain.

It is further contemplated that the mRNA could comprises a sequence encoding a bacterial antigen, but having a 5′ cap or pseudo cap (i.e. IRES element) and a 3′ poly-A sequence along with the sequence encoding the bacterial antigen or fragment thereof. As such, this would be a non-naturally occurring eukaryote-translatable mRNA encoding a bacterial polypeptide.

The eukaryote-translatable mRNA may be, for example, one encoding a protein having some function such as enzyme, receptor, transporter, antibody, structural protein, and regulator, or one encoding a protein having no function per se. Incidentally, the term “protein” referred to herein includes so-called peptides such as oligopeptide and polypeptide.

The length of the eukaryote-translatable mRNA is not limited. The length of the eukaryote-translatable mRNA, for example, may be 10 nucleotides or more, 20 nucleotides or more, 50 nucleotides or more, or 100 nucleotides or more, or 10000 nucleotides or more, or may be 10000 nucleotides or less, 5000 nucleotides or less, 2000 nucleotides or less, 1000 nucleotides or less, or 500 nucleotides or less, or may be a range defined as a combination thereof.

The eukaryote-translatable mRNA is single-stranded RNA and may be, for example, one molecule of RNA in a linear or circular (i.e., covalently closed) conformation.

In one embodiment of this invention the eukaryote-translatable mRNA is circularized in the bacteria upon its transcription. For example, a bacteriophage T4 permuted intron-exon (PIE) method can be used to promote circularization of the mRNA. Through group I intron self-splicing, splicing and then ligation of two exons occurs forming a circular RNA product which can theoretically be translated inside a eukaryotic cell. The circular mRNA may in some instances be transcribed with a 3′ poly-A sequence. A circular mRNA conformation may in some instances prove advantageous in that the 5′ and 3′ ends are inaccessible to RNases, thereby preventing degradation of the mRNA molecule and enhancing stability of the eukaryote-translatable mRNA.

In the present invention it may be important in some instances to inhibit prokaryotic translation initiation of the eukaryote-translatable mRNA. Methods of inhibiting prokaryotic translation include but are not limited to eliminating any sequence recognized as a unit as a bacterial ribosome binding site (RBS); eliminating the epsilon sequence element (UUAACUUUA), a translational enhancer, or the like; deleting or mutating sequences upstream of the eukaryote-translatable RNA cassette that are identical to or otherwise recognized as a Shine-Dalgarno (SD) sequence; or any other method of preventing prokaryotic translation of the eukaryote-translatable mRNA.

More specifically, the formation of the protein from the eukaryote-translatable mRNA transcript can be preferably prevented by partially or completely deleting or mutating the bacterial ribosome binding site (RBS), which includes a Shine-Dalgarno (SD) sequence or other sequence that functions in the same capacity, in the vector used for transcribing the eukaryote-translatable mRNA so that the formed eukaryote-translatable mRNA will not be translated in the bacterial cell due to the absence of a functional prokaryotic ribosomal binding site (RBS), which is required to bind the ribosome and initiate translation of the RNA to the encoded protein. In bacteria, the consensus Shine-Dalgarno (SD) sequence is known to be AGGAGG. In E. coli, the sequence is known to be AGGAGGU or variations thereof that serve the same function in prokaryotic translation initiation. In other bacterial species (e.g., Corynebacteria), sequences varying from the consensus can also serve the same function in translation initiation.

In another embodiment, because the eukaryote-translatable mRNA is an mRNA that is to be translated in eukaryotic cells, the vector used for transcribing the eukaryote-translatable RNA may include a Kozak sequence, which is necessary for ribosome binding in eukaryotic cells.

The term “expression unit for eukaryote-translatable mRNA” refers to a genetic construct (e.g., vector) configured so that the eukaryote-translatable mRNA can be transcribed therefrom. The expression unit for the eukaryote-translatable mRNA contains a promoter sequence that functions in a prokaryote and a nucleotide sequence encoding the eukaryote-translatable mRNA in the direction from 5′ to 3′. The promoter sequence is also simply referred to as “promoter”.

In another embodiment of the present invention, the expression unit for eukaryote-translatable mRNA contains a promoter sequence that functions in a eukaryote, a nucleotide sequence encoding the eukaryote-translatable mRNA in the direction from 5′ to 3′, and additional nucleotide sequences (e.g., bacteriophage T4 PIE sequences included upstream and downstream of the eukaryote-translatable mRNA expression unit) which may promote formation of a circularized RNA transcript. Promoters can include, but are not limited to, CMV, SV40, H1, PGK1, EF1a, and U6.

The term “expression” or “expressing” of eukaryote-translatable mRNA refers to the transcription of the eukaryote-translatable mRNA by the bacterial cell.

The nucleotide sequence encoding the eukaryote-translatable mRNA is also referred to as the “gene encoding eukaryote-translatable mRNA” or “eukaryote-translatable mRNA gene”. In one embodiment, the eukaryote-translatable mRNA gene is present downstream of a prokaryotic promoter so that the eukaryote-translatable mRNA is expressed under control of said promoter. The expression unit for the eukaryote-translatable mRNA may also contain regulatory sequence(s) effective for expressing the eukaryote-translatable mRNA in a bacterium; such sequences include but are not limited to RNA polymerase binding sites (e.g., −35 and −10 sequences), which may or may not be specific for a specific RNA polymerase sigma subunit, UP elements (sequences that interact with the RNA polymerase alpha subunit), operator sequences, and terminator sequences at appropriate position(s) so that the regulatory sequence(s) can function. The expression unit for the eukaryote-translatable mRNA can be appropriately designed according to various conditions such as the transcription pattern of the eukaryote-translatable mRNA.

In some instances, it may be desirable that the nucleotide sequence encoding the eukaryote-translatable mRNA is codon optimized for eukaryotic translation.

The eukaryote-translatable mRNA associated with a particular gene can be obtained prior to ligation downstream of a promoter by, for example, by cloning or nucleotide synthesis.

In one embodiment of the invention, the promoter for expressing the eukaryote-translatable mRNA gene functions in the bacterium. The “promoter that functions in a bacterium” refers to a promoter that shows a promoter activity, i.e., transcription promoting activity, in the bacterium. The promoter may be a promoter derived from the bacterium or a heterologous promoter. The promoter may be the native promoter of the eukaryote-translatable mRNA gene, or a promoter of another gene. The promoter may be an inducible promoter or a constitutive promoter for gene expression.

In alternative embodiment of the invention, when the eukaryote-translatable mRNA is to be expressed to a eukaryotic cell, the promoter for expressing the eukaryote-translatable mRNA-encoding gene may be a promoter (e.g. a eukaryotic promoter) that functions in the eukaryotic host.

In one embodiment, the bacterium of the present invention may contain the expression unit for the eukaryote-translatable mRNA within a vector autonomously replicable separately from the chromosome, such as a plasmid, cosmid, bacterial artificial chromosome, bacteriophage, or any extrachromosomal element, or the expression unit may be integrated into the chromosome. In other words, the bacterium of the present invention, for example, may have the expression unit for the eukaryote-translatable mRNA on a vector, and may have a vector containing the expression unit for the eukaryote-translatable mRNA. The bacterium of the present invention, for example, may also have the expression unit for the eukaryote-translatable mRNA on the bacterial chromosome. The vector preferably contains a marker such as an antibiotic resistance gene, auxotrophy-complementing gene, or antibiotic-independent mechanism for vector maintenance and for selection of transformants. The mechanism for vector maintenance may be, for example, accomplished using a bacteriocin such as microcin V or other bacteriocin-based vector selection.

The bacterium of the present invention may have one or more copies of the expression unit for the eukaryote-translatable mRNA. The copy number of the expression unit for the eukaryote-translatable mRNA possessed by the bacterium of the present invention, for example, may be as few as 1 copy/cell (e.g. as an integration into the bacterial chromosome) or more than 2000 copies/cell (e.g. via cloning into plasmids of varying replication origins to alter copy number) or may be a range defined as a non-contradictory combination thereof. The bacterium of the present invention may have one kind/type of expression unit or more than one kind/type of expression unit for the eukaryote-translatable mRNA per cell.

The copy number and kind/type of the expression unit for the eukaryote-translatable mRNA may also be read as the copy number and kind/type of the eukaryote-translatable mRNA gene, respectively. When the bacterium of the present invention has two or more expression units for the eukaryote-translatable mRNA, it is sufficient that those expression units are harbored by the bacterium of the present invention so that the eukaryote-translatable mRNA is produced. In other words, all said expression units may be harbored on a single expression vector or on the chromosome. Alternatively, those expression units may be harbored separately on a plurality of expression vectors, or separately on a single or plurality of expression vectors and the chromosome.

The bacterium of the present invention can be cultured under such conditions so that the eukaryote-translatable mRNA is transcribed and accumulated in the bacterial cells. For example, the bacterium can be incubated at 37° C. in a nutritionally rich growth medium (e.g., brain heart infusion medium), and cultured to the exponential growth phase wherein the eukaryote-translatable mRNA is constitutively transcribed and continuously accumulating within each bacterial cell throughout the incubation period.

The expression and accumulation of the eukaryote-translatable mRNA can be confirmed by, for example, by a molecular method such as PCR or nucleotide sequencing, or by applying a bacterial cell extract as a sample to electrophoresis and subsequently detecting a band corresponding to the molecular weight of the eukaryote-translatable mRNA.

The term “collected from the cells” also means extracted from the bacterium producing the eukaryote-translatable mRNA. In some instances, it may be desirable to treat the bacterial culture broth with an RNA protection reagent to stabilize the mRNA inside the bacteria and promote mRNA stabilization prior to mRNA collection procedures. The RNA protection reagent may be produced exogenously and added to the bacteria or it may be produced by the bacteria themselves.

The eukaryote-translatable mRNA containing an IRES element (in place of a 5′ cap) and a poly-A tail can be collected from the bacterial cells by appropriate methods used for separation and purification of such compounds. In a preferred embodiment of the present invention the eukaryote-translatable mRNA is obtained from the bacterial cell by separating the target eukaryote-translatable mRNA from the endogenous RNA of the bacterial cell.

Examples of such collection methods include but are not limited to any combination of salting out, gel filtration chromatography, centrifugation, ethanol precipitation, ultrafiltration, ion exchange chromatography, affinity chromatography, and electrophoresis. Specifically, for example, the bacterial cells can be disrupted with ultrasonic waves and a supernatant can be obtained by removing the bacteria from the disrupted cell suspension by centrifugation or the like, and the eukaryote-translatable mRNA can be collected from the supernatant by the ion exchange resin method or a similar method. The collected eukaryote-translatable mRNA may be a free compound, a salt thereof, or a mixture thereof. In addition, the collected eukaryote-translatable mRNA may also be a complex with a high-molecular-weight compound such as a protein. That is, in the present invention, the term “eukaryote-translatable mRNA” may refer to the eukaryote-translatable mRNA in a free form, a salt thereof, a complex thereof with a high-molecular-weight compound such as a protein, or a mixture thereof, unless otherwise stated. Examples of the salt include, for example, ammonium salt and sodium salt.

In one embodiment, the step of obtaining the eukaryote-translatable mRNA comprises a step of depleting the ribosomal RNA of the bacterial cell and more preferably the ribosomal RNA of the bacterial cell is depleted by capture hybridization of the ribosomal RNA with complementary oligonucleotides immobilized on a solid phase. Another example of obtaining the eukaryote-translatable mRNA is through RNase H-based enzymatic depletion methods.

In a preferred embodiment of the present invention the eukaryote-translatable mRNA is obtained by hybridization with a complementary nucleic acid sequence.

In a specific embodiment of the present invention the complementary nucleic acid sequence is immobilized on a solid matrix.

In one embodiment of this invention, the collected eukaryote-translatable mRNA can be stored for downstream applications. Storage formulations may include, for example, as a lyophilized or freeze-dried product with or without stabilizers or excipients.

The collected eukaryote-translatable mRNA may contain, for example, such components as bacterial cells, medium components, moisture, and by-product metabolites of the bacterium, in addition to the eukaryote-translatable mRNA. The eukaryote-translatable mRNA may also be purified at a desired extent. Purity of the collected eukaryote-translatable mRNA may be, for example, 30% (w/w) or higher, 50% (w/w) or higher, 70% (w/w) or higher, 80% (w/w) or higher, 90% (w/w) or higher, or 95% (w/w) or higher.

In the present invention, the bacteria producing the eukaryote-translatable mRNA contain at least one expression cassette encoding the eukaryote-translatable mRNA, on a plasmid, cosmid, bacterial artificial chromosome, bacteriophage or the bacterial chromosome (all also referred to as vector); the eukaryote-translatable mRNA may contain a bacterially transcribed poly-A region, and a 5′ cap or pseudo-cap element, e.g., an internal ribosome entry site (IRES) element, that mediates translation in the eukaryotic host cell. Examples of possible IRES elements are found in Tables 1, 2 and 3. Additional IRES elements include any IRES elements that are effective at eukaryotic ribosome recruitment and translation initiation but minimally effective for the same in prokaryotes.

A DNA sequence that “encodes” a particular RNA is a DNA nucleic acid sequence that is transcribed into RNA. A DNA polynucleotide may encode an RNA (mRNA) that is translated into protein, or a DNA polynucleotide may encode an RNA that is not translated into protein (e.g. tRNA, rRNA, or a guide RNA; also called “non-coding” RNA or “ncRNA”). A “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence will usually be located 3′ to the coding sequence.

As used herein, a “promoter” or “promoter sequence” is a DNA regulatory region capable of binding RNA polymerase and initiating transcription of a downstream (3′ direction) coding or non-coding sequence. For purposes of defining the present invention, the promoter sequence is bounded at its 3″ terminus by the transcription initiation site and extends upstream (5′ direction) to include the minimum number of bases or elements necessary to initiate transcription at levels detectable above background. Within the promoter sequence a transcription initiation site will be found, as well as protein binding domains responsible for the binding of RNA polymerase. Various promoters, including inducible promoters, may be used to drive the vectors as described in the present disclosure.

A promoter can be a constitutively active promoter (i.e., a promoter that is constitutively in an active (“ON”) state), it may be an inducible promoter (i.e., a promoter whose state, active (“ON”) or inactive (“OFF”), is controlled by an external stimulus, (e.g., the presence of a particular temperature, compound, or protein).

As used herein, the term “invasive” when referring to a microorganism, e.g., a bacterium or bacterial therapeutic particle (BTP), refers to a microorganism that is capable of delivering at least one molecule, e.g., an RNA or RNA-encoding DNA molecule, or eukaryote-translatable mRNA, to a target cell. An invasive microorganism can be a microorganism that is capable of traversing a cell membrane, thereby entering the cytoplasm of said cell, and delivering at least some of its content, e.g., RNA or RNA-encoding DNA, into the target cell. The process of delivery of the at least one molecule into the target cell preferably does not significantly modify the invasion apparatus.

As used herein, the term “transkingdom” refers to a delivery system that uses bacteria (or another invasive microorganism) to generate nucleic acids and deliver the nucleic acids intracellularly (i.e. across kingdoms: prokaryotic to eukaryotic, or across phyla: invertebrate to vertebrate) within target tissues for processing without host genomic integration.

Invasive microorganisms include microorganisms that are naturally capable of delivering at least one molecule to a target cell, such as by traversing the cell membrane, e.g., a eukaryotic cell membrane, and entering the cytoplasm, as well as microorganisms which are not naturally invasive and which have been modified, e.g., genetically modified, to be invasive. In another preferred embodiment, a microorganism that is not naturally invasive can be modified to become invasive by linking the bacterium or BTP to an “invasion factor”, also termed “entry factor” or “cytoplasm-targeting factor”. As used herein, an “invasion factor” is a factor, e.g., a protein or a group of proteins which, when expressed by a non-invasive bacterium or BTP, render the bacterium or BTP invasive. As used herein, an “invasion factor” is encoded by a “cytoplasm-targeting gene”. Invasive microorganisms have been generally described in the art, for example, U.S. Pat. Pub. Nos. US 20100189691 A1 and US20100092438 A1 and Xiang, S. et al., Nature Biotechnology 24, 697-702 (2006). Each of which is incorporated by reference in its entirety for all purposes.

In a preferred embodiment the invasive microorganism is E. coli, as taught in the examples of the present application. However, it is contemplated that additional microorganisms could potentially be adapted to perform as transkingdom delivery vehicles for the delivery of gene-editing cargo. These non-virulent and invasive bacteria and BTPs would exhibit invasive properties, or would be modified to exhibit invasive properties, and may enter a host cell through various mechanisms. In contrast to uptake of bacteria or BTPs by professional phagocytes, which normally results in the destruction of the bacterium or BTP within a specialized lysosome, invasive bacteria or BTP strains have the ability to invade non-phagocytic host cells. Naturally occurring examples of such intracellular bacteria are Yersinia, Rickettsia, Legionella, Brucella, Mycobacterium, Helicobacter, Coxiella, Chlamydia, Neisseria, Burkolderia, Bordetella, Borrelia, Listeria, Shigella, Salmonella, Staphylococcus, Streptococcus, Porphyromonas, Treponema, and Vibrio, but this property can also be transferred to other bacteria or BTPs such as E. coli, Lactobacillus, Lactococcus, or Bifidobacteriae, including probiotics through the transfer of invasion-related genes (P. Courvalin, S. Goussard, C. Grillot-Courvalin, C. R. Acad. Sci. Paris 318, 1207 (1995)). Factors to be considered or addressed when evaluating additional bacterial species as candidates for use as transkingdom delivery vehicles include the pathogenicity, or lack thereof, of the candidate, the tropism of the candidate bacteria for the target cell, or, alternatively, the degree to which the bacteria can be engineered to deliver gene-editing cargo to the interior of a target cell, and any synergistic value that the candidate bacteria might provide by triggering the host's innate immunity.

As used herein the term “fully functional mRNA” or “functional mRNA” refers to RNA molecules that contains a 3′ transcribed poly-A region and a 5′ cap or pseudo-cap element, e.g., an internal ribosome entry site (IRES) element, so that a eukaryotic ribosome translates the mRNA into a polypeptide.

As used herein the term “eukaryote-translatable element” refers to mRNA that contains a poly-A sequence transcribed by the bacteria and a 5′ cap or pseudo-cap element, e.g., an internal ribosome entry site (IRES) element, that mediates ribosome recruitment and translation in the eukaryotic host cell. The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

The methods of administering these improved transkingdom NA delivery vehicles include intranasal dosing to nasal cavity for local action, aerosolization for upper and lower respiratory targeting, absorption in the oral cavity for buccal delivery, ingestion for GI adsorption, application to delicate genital mucosal epithelium, and topical administration for ocular delivery. These improved delivery vehicles could be used to prevent and/or treat a wide range of diseases (infectious, allergic, cancerous, and immunological) in a wide range of species (human, avian, swine, bovine, canine, equine, feline).

The term “administration” and variants thereof (e.g., “administering” a compound) in reference to a compound of the invention means introducing the compound into the system of the subject in need of treatment. When a compound of the invention is provided in combination with one or more other active agents (e.g., a cytotoxic agent, etc.), “administration” and its variants are each understood to include concurrent and sequential introduction of the compound and other agents.

A “subject” is any multi-cellular vertebrate organism, such as human and non-human mammals (e.g., veterinary subjects). In one example, a subject is known or suspected of having an infection or other condition that is life-threatening or impairs the quality of life.

The terms “treating” and “treatment” as used herein refer to the administration of an agent or formulation (e.g., bacterium) of the invention to a clinically symptomatic subject afflicted with an adverse condition, disorder, or disease, so as to effect a reduction in severity and/or frequency of symptoms, eliminate the symptoms and/or their underlying cause, and/or facilitate improvement or remediation of damage.

The terms “preventing” and “prevention” refer to the administration of an agent or composition to a clinically asymptomatic individual who is susceptible to a particular adverse condition, disorder, or disease, and thus relates to the prevention of the occurrence of symptoms and/or their underlying cause.

Invasive bacteria containing the mRNA can be introduced into a subject by intravenous, intramuscular, intradermal, intraperitoneally, peroral, intranasal, intraocular, intrarectal, intravaginal, intraosseous, oral, immersion and intraurethral inoculation routes. The amount of the invasive bacteria of the present invention to be administered to a subject will vary depending on the species of the subject, as well as the disease or condition that is being treated. For example, a dosage could be about 10³ to 10¹¹ viable organisms, preferably about 10⁵ to 10⁹ viable organisms per subject. The invasive bacteria or BTPs of the present invention are generally administered along with a pharmaceutically acceptable carrier and/or diluent.

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

For administration by inhalation, the pharmaceutical compositions for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the composition, e.g., bacteria, and a suitable powder base such as lactose or starch.

The pharmaceutical compositions may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water,

The invasive bacteria containing the mRNA to be introduced can be used to infect animal cells that are cultured in vitro, such as cells obtained from a subject. These in vitro-infected cells can then be introduced into animals, e.g., the subject from which the cells were obtained initially, intravenously, intramuscularly, intradermally, or intraperitoneally, or by any inoculation route that allows the cells to enter the host tissue. When delivering RNA to individual cells, the dosage of viable organisms administered will be at a multiplicity of infection ranging from about 0.1 to 10⁶, preferably about 10² to 10⁴ bacteria per cell. In yet another embodiment of the present invention, bacteria can also deliver mRNA molecules encoding proteins to cells, e.g., animal cells, from which the proteins can later be harvested or purified. For example, a protein can be produced in a tissue culture cell.

Six tables are presented below.

TABLE 1 provides examples of possible non-human eukaryotic IRES elements. The gene indicated by the given gene symbol is known to encode an associated, specific IRES sequence that controls the translation of said gene's RNA transcript. IRES elements are discussed more fully in the literature [see e.g. A Bioinformatical Approach to the Analysis of Viral and Cellular Internal Ribosome Entry Sites. In: Columbus F editors. New Messenger RNA Research Communications. Hauppauge, N.Y.: Nova Science Publishers; pp. 133-166 (2007); Mokrejs M, Vopálenský V, Kolenaty O, Masek T, Feketová Z, Sekyrová P, Skaloudová B, Kriz V, Pospisek M. IRESite: the database of experimentally verified IRES structures (www.iresite.org). Nucleic Acids Res. 2006 Jan. 1; 34(Database issue):D125-30. doi: 10.1093/nar/gkj081. PMID: 16381829; PMCID: PMC 1347444.

TABLE 2 provides examples of possible viral IRES elements. The virus indicated by the given virus symbol is known to encode one associated, specific IRES sequence that controls the translation of said virus' RNA transcript.

TABLE 3 provides examples of possible human IRES elements. The gene indicated by the gene symbol encodes an IRES element in the 5′ end of the RNA.

TABLE 6 provides the sequences for select viral IRES sequences. Included are three viral IRES elements and an additional (optional) sequence for using the CrPV viral IRES with a circular transcript, which includes the sequence that allows for circularization of the RNA.

All references cited in the present application are incorporated in their entirety herein by reference to the extent not inconsistent herewith.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

TABLE 1

| Organism | Gene Symbol |
|---|---|
| Aplysia californica (California sea hare) | ELH |
| Canis lupus familiaris (dog) | SCAMPER |
| Drosophila melanogaster (fruit fly) | Antp |
| Drosophila melanogaster (fruit fly) | Hsp70Aa |
| Drosophila melanogaster (fruit fly) | Hsp83 |
| Drosophila melanogaster (fruit fly) | rpr |
| Drosophila melanogaster (fruit fly) | Ubx |
| Drosophila melanogaster (fruit fly) | gag |
| Drosophila melanogaster (fruit fly) | mbl |
| Drosophila melanogaster (fruit fly) | Pde8 |
| Drosophila melanogaster (fruit fly) | cdi |
| Drosophila melanogaster (fruit fly) | tai |
| Drosophila melanogaster (fruit fly) | CG5460; Dmel\CG5460; |
| | HFL |
| Drosophila melanogaster (fruit fly) | hid |
| Drosophila melanogaster (fruit fly) | grim |
| Drosophila melanogaster (fruit fly) | InR |
| Drosophila melanogaster (fruit fly) | foxo |
| Drosophila melanogaster (fruit fly) | Adh-dup |
| Gallus gallus (chicken) | JUN |
| Mus musculus (house mouse) | Nkx6-2 |
| Mus musculus (house mouse) | Hif1a |
| Mus musculus (house mouse) | Kcna4 |
| Mus musculus (house mouse) | Ndst1 |
| Mus musculus (house mouse) | Ndst2 |
| Mus musculus (house mouse) | Ndst3 |
| Mus musculus (house mouse) | Ndst4 |
| Mus musculus (house mouse) | |
| Mus musculus (house mouse) | Vegfa |
| Mus pahari (shrew mouse) | Utrn |
| Mus musculus (house mouse) | Odc1 |
| Mus musculus (house mouse) | Gja1 |
| Mus musculus (house mouse) | Gjb1 |
| Mus musculus (house mouse) | Hr |
| Mus musculus (house mouse) | Rbm3 |
| Mus musculus (house mouse) | Shmt1 |
| Mus musculus (house mouse) | Cirbp |
| Mus musculus (house mouse) | Rev-erb [symbol] ± |
| Nicotiana tabacum (common tobacco) | LOC107810899 |
| Rattus norvegicus (Norway rat) | Slc7a1 |
| Saccharomyces cerevisiae (baker's yeast) | YAP1 |
| Saccharomyces cerevisiae (baker's yeast) | TIF4631 |
| Saccharomyces cerevisiae S288C | URE2 |
| Saccharomyces cerevisiae (baker's yeast) | HAP4 |
| Saccharomyces cerevisiae (baker's yeast) | TFIID |
| Saccharomyces cerevisiae S288C | YMR181c |
| Saccharomyces cerevisiae (baker's yeast) | BOI1 |
| Saccharomyces cerevisiae (baker's yeast) | FLO8 |
| Saccharomyces cerevisiae S288C | GIC1 |
| Saccharomyces cerevisiae S288C | MSN1 |
| Saccharomyces cerevisiae S288C | NCE102 |
| Saccharomyces cerevisiae S288C | GPR1 |
| Zea mays | HSP101 |
| Zea mays | adh1 |
| Mus musculus (house mouse) | Fmr1 |
| Drosophila melanogaster (fruit fly) | HFL |
| Drosophila melanogaster (fruit fly) | Hp121 |
| Drosophila melanogaster (fruit fly) | Drs |
| Drosophila melanogaster (fruit fly) | AttA |
| Drosophila melanogaster (fruit fly) | Thor |
| Rattus norvegicus (Norway rat) | Sp1 |
| Rattus norvegicus (Norway rat) | Aanat |
| Rattus norvegicus (Norway rat) | Nrgn |
| Rattus norvegicus (Norway rat) | Camk2a |
| Rattus norvegicus (Norway rat) | Ddn |
| Rattus norvegicus (Norway rat) | Map2 |
| Rattus norvegicus (Norway rat) | Arc |
| Rattus norvegicus (Norway rat) | Prkcd |
| Rattus norvegicus (Norway rat) | Avpr1b |
| Ovis aries (sheep) | AANAT |
| Mus musculus (house mouse) | Vegfd |
| Mus musculus (house mouse) | Ahr |
| Mus musculus (mouse) | Per1 |
| Mus musculus (house mouse) | PEBP2a |
| Mus musculus (house mouse) | Runx2 |
| Mus musculus (house mouse) | TrkB (Ex1a) |
| Mus musculus (house mouse) | Ntrk2 |
| Mus musculus (house mouse) | Rnf2 |
| Saccharomyces cerevisiae | Hansenula |
| | polymorpha DL-1 |

TABLE 2
Virus Name

Human betaherpesvirus 5 (HHV-5; HCMV)
Kashmir bee virus (KBV)
Homalodisca coagulata virus-1 (HoCV-1)
Human alphaherpesvirus 1 (Herpes simplex virus 1, HHV-1)
Ovine enzootic nasal tumor virus
Black queen cell virus (BQCV)
Human papillomavirus type 31 (HPV31)
Human adenovirus 7 (HAdV7)
Human mastadenovirus D (HAdV-D)
Human adenovirus 5 (HAdV5)
Human adenovirus 54 (HAdV-54)
Human papillomavirus type 4 (HPV4)
Macaca mulatta polyomavirus 1
Human betaherpesvirus 6B (HHV-6B)
Human coronavirus OC43 (HCoV-OC43)
Human papillomavirus type 53 (HPV53)
Human betaherpesvirus 6A (HHV-6A)
Human gammaherpesvirus 4 (Epstein-Barr virus);
Human herpesvirus 4 type 2 (Epstein-Barr virus type 2)
Xenopus laevis endogenous retrovirus Xen1
Human alphaherpesvirus 2 (Herpes simplex virus 2, HHV-2)
Human gammaherpesvirus 4 (Epstein-Barr virus)
Human betaherpesvirus 7 (HHV-7)
Human papillomavirus type 41 (HPV4)
Human mastadenovirus E (HAdV-E)
Pigeon picornavirus B
Human papillomavirus type 16 (HPV16)
Human papillomavirus type 50 (HPV50)
Human gammaherpesvirus 8 (Kaposi's
sarcoma-associated herpesvirus)
Human alphaherpesvirus 3 (HHV-3)
Taura syndrome virus (TSV)
Human mastadenovirus B (HAdV-B)
Hepatovirus A
Human mastadenovirus A
Human papillomavirus type 48 (HPV48)
Human papillomavirus type 92 (HPV92)
Hepatitis C virus (HCV)
Human papillomavirus type 34 (HPV34)
Human papillomavirus type 49 (HPV49)
Feline leukemia virus (FeLV)
Feline picornavirus
Modoc virus (MODV)
Human papillomavirus type 96 (HPV96)
Human papillomavirus type 90 (candHPV90)
Human mastadenovirus F (HAdV-F)
Human mastadenovirus C; Human adenovirus 2 (HAdV-C)
Macaque simian foamy virus (SFVmac)
Duck hepatitis A virus 3 (DHAV-3)
Tai Forest ebolavirus
Mason-Pfizer monkey virus (M-PMV)
Human papillomavirus type 7 (HPV7)
Human papillomavirus type 10 (HPV10)
Human papillomavirus type 9 (HPV9)
Human metapneumovirus (HMPV)
Human papillomavirus type 101 (HPV101)
Human herpesvirus 4 type 2 (Epstein-Barr virus type 2)
Human papillomavirus type 5 (HPV5)
Bundibugyo ebolavirus
Zaire ebolavirus (ZEBOV)
Snakehead retrovirus (SnRV)
Spleen focus-forming virus (SFFV)
Human herpesvirus 8 type M (HHV-8M)
African green monkey simian foamy virus
Acute bee paralysis virus
Human mastadenovirus B (HAdV-B); Human
adenovirus 7 (HAdV7)
Human poliovirus 3
Human papillomavirus type 63 (HPV63)
Human mastadenovirus C; Human adenovirus 2;
Human adenovirus 5 (HAdV-C)
Rhopalosiphum padi virus (RhPV)
Human papillomavirus type 60 (HPV60)
Human immunodeficiency virus 2 (HIV-2)
Rotavirus C
Human papillomavirus type 32 (HPV32)
Drosophila C virus (DCV)
Triatoma virus (TrV)
Bovine viral diarrhea virus 1 (BVDV-1)
Sudan ebolavirus
Rabies lyssavirus
Simian T-lymphotropic virus 1 (STLVs-1)
Human coronavirus HKU1 (HCoV-HKU1)
Mouse mammary tumor virus (MMTV)
Foot-and-mouth disease virus - type
SAT 2 (FMDV-SAT2)
Solenopsis invicta virus-1 (SINV-1)
Simian immunodeficiency virus (SIV)
GB virus C (GBV-HGV)
Human coronavirus NL63 (HCoV-NL63)
Human papillomavirus type 11 (HPV11)
Human papillomavirus type 88 (HPV88)
Canine picodicistrovirus
Cricket paralysis virus (CrPV)
Human enterovirus 107 (HEV-107)
Rotavirus A
Banna virus strain JKT-6423
Tremovirus A
Turkey gallivirus
Human papillomavirus type 26 (HPV26)
Machupo mammarenavirus
Human parvovirus 4 G1
West Nile virus (WNV)
Hepatitis GB virus B (HGBV-B)
Theilovirus (ThV)
Classical swine fever virus (CSFV)
Feline immunodeficiency virus (FIV)
Feline foamy virus (FFV/FeFV)
Human coronavirus 229E (HCoV-229E)
Nipah henipavirus (NiV)
Human immunodeficiency virus 1 (HIV-1)
Hendra henipavirus (HeV)
Human papillomavirus type 108 (HPV108)
Alphapapillomavirus 4
Pelargonium flower break virus (PFBV)
Plautia stali intestine virus (PSIV)
Enterovirus J (simian virus 6)
Abelson murine leukemia virus (A-MuLV)
Human polyomavirus 1
Enterovirus J
Human parvovirus B19
Ovine lentivirus (OLV/OvLV)
Squirrel monkey retrovirus (SMRV)
Human papillomavirus type 103 (HPV103)
Israeli acute paralysis virus (IAPV)
Giardia lamblia virus (GLV)
Pegivirus A
Aphid lethal paralysis virus (ALPV)
Human erythrovirus V9 (HEV-V9)
Lloviu cuevavirus
Enzootic nasal tumour virus of goats (ENTV-2)
Bovine foamy virus (BFV)
Human papillomavirus type 6b (HPV6b)
Influenza B virus (B/Lee/1940)
Jaagsiekte sheep retrovirus (JSRV)
Rauscher murine leukemia virus
Human bocavirus 4 NI (HBoV4-NI)
Moloney murine leukemia virus
Alphapapillomavirus 7
Coxsackievirus B3 (CVB3)
Human foamy virus (HFV)
Human enterovirus 100 (HEV-100)
Simian T-cell lymphotropic virus 6 (STLVs-6)
Foot-and-mouth disease virus - type A (FMDV-A)
Equine foamy virus (EFV)
Marburg marburgvirus
Ectropis obliqua picorna-like virus (EoPV)
Mud crab dicistrovirus (MCDV)
Tobamovirus
Aichi virus 1 (AiV-1)
Encephalomyocarditis virus (EMCV)
Rhinovirus A
Human T-lymphotropic virus 2 (HTLV-2)
Hepatitis B virus (HBV)
Influenza A virus (A/goose/Guangdong/1/1996(H5N1))
Equine rhinitis A virus (ERAV)
Simian retrovirus 4 (SRV-4)
Enterovirus A71 (EV-A71)
Murine osteosarcoma virus
Woolly monkey sarcoma virus (WMSV)
Human respirovirus 1 (HPIV-1)
Reticuloendotheliosis virus (REV)
Porcine teschovirus 1 (PTV-1)
Rous sarcoma virus (RSV)
Simian immunodeficiency virus SIV-mnd 2 (SIVmnd-2)
Human poliovirus 2
Equine rhinitis B virus 1 (ERBV-1)
Friend murine leukemia virus
Enterovirus A
Himetobi P virus (HiPV)
Influenza A virus (A/New York/392/2004(H3N2))
Simian enterovirus 19 (simian virus 19)
Gallid alphaherpesvirus 2 (Marek's
disease virus type 1)
Bovine hungarovirus 1 (BHuV-1)
Human gammaherpesvirus 4
Human gammaherpesvirus 8
Turnip vein-clearing virus
Turnip crinkle virus
Human poliovirus 1
Senecavirus A
Human immunodeficiency virus 2
Murine leukemia virus
Human betaherpesvirus 5
Blackcurrant reversion virus
Hibiscus chlorotic ringspot virus
Potato leafroll virus
Tobacco etch virus
Leishmania RNA virus 1-4
Hepacivirus N
Triticum mosaic virus
Human T-cell leukemia virus type I
Cloning vector pGR102
HIV-1 vector pNL4-3
Infectious pancreatic necrosis virus
Equine hepacivirus JPN3/JAPAN/2013
Rosellinia necatrix victorivirus 1
Helminthosporium victoriae virus 190S
Helminthosporium victoriae 145S virus
Cryphonectria nitschkei chrysovirus 1
Dengue virus 2
Zika virus
Infectious flacherie virus
Simian sapelovirus 3
Gallid alphaherpesvirus 2
Leishmania RNA virus 1 - 1
Cryphonectria hypovirus 1
Cryphonectria hypovirus 2-NB58
Cryphonectria hypovirus 3

TABLE 3

| Gene | |
|---|---|
| Symbol | Gene Synonym |
| ABCF1 | ABC27; ABC50 |
| ABCG1 | ABC8; WHITE1 |
| ACAD10 | |
| ACOT7 | ACH1; ACT; BACH; CTE-II; |
| | hBACH; LACH; LACH1 |
| ACSS3 | |
| ACTG2 | ACT; ACTA3; ACTE; ACTL3; |
| | ACTSG; VSCM |
| ADCYAP1 | PACAP |
| ADK | AKADK |
| AGTR1 | AG2S; AGTR1B; AT1; AT1AR; |
| | AT1B; AT1BR; AT1R; |
| | AT2R1; HAT1R |
| AHCYL2 | ADOHCYASE3; IRBIT2 |
| AHI1 | AHI-1; dJ71N10.1; JBTS3; |
| | ORF1 |
| AKAP8L | HA95; HAP95; NAKAP; NAKAP95 |
| AKR1A1 | ALDR1; ALR; ARM; DD3; HEL-S-6 |
| ALDH3A1 | ALDH3; ALDHIII |
| ALDOA | ALDA; GSD12; HEL-S-87p |
| ALG13 | CDG1S; CXorf45; EIEE36; |
| | GLT28D1; MDS031; |
| | TDRD13; YGL047W |
| AMMECR1L | |
| ANGPTL4 | ARP4; FIAF; HARP; HFARP; |
| | NL2; PGAR; pp1158; |
| | TGQTL; UNQ171 |
| ANK3 | ANKYRIN-G; MRT37 |
| AOC3 | HPAO; SSAO; VAP-1; VAP1 |
| AP4B1 | BETA-4; CPSQ5; SPG47 |
| AP4E1 | CPSQ4; SPG51; STUT1 |
| APAF1 | APAF-1; CED4 |
| APBB1 | FE65; MGC:9072; RIR |
| APC | BTPS2; DP2; DP2.5; DP3; |
| | GS; PPP1R46 |
| APH1A | 6530402N02Rik; APH-1; |
| | APH-1A; CGI-78 |
| APOBEC3D | A3D; APOBEC3DE; APOBEC3E; |
| | ARP6 |
| APOM | apo-M; G3a; HSPC336; NG20 |
| APP | AAA; AD1; CVAPAPP; ABETA |
| AQP4 | MIWC; WCH4 |
| ARHGAP36 | |
| ARL13B | ARL2L1; JBTS8 |
| ARMC8 | GID5; HSPC056; S863-2; |
| | VID28 |
| ARMCX6 | GASP10 |
| ARPC1A | Arc40; HEL-68; HEL-S-307; |
| | SOP2Hs; SOP2L |
| ARPC2 | AD022; dJ30M3.3; EAP2; |
| | EAPII; hTDP2; TTRAP |
| ARRDC3 | TLIMP |
| ASAP1 | AMAP1; CENTB4; DDEF1; |
| | PAG2; PAP; ZG14P |
| ASB3 | ASB-3 |
| ASB5 | ASB-5 |
| ASCL1 | ASH1; bHLHa46; HASH1; |
| | MASH1 |
| ASMTL | ASMTLX; ASMTLY; ASTML |
| ATF2 | CRE-BP1; CREB-2; CREB2; |
| | HB16; TREB7 |
| ATF3 | |
| ATG4A | APG4A; AUTL2 |
| ATP5B | ATPMB; ATPSB; HEL-S-271 |
| ATP6V0A1 | a1; ATP6N1; ATP6N1A; Stv1; |
| | Vph1; VPP1 |
| ATXN3 | AT3; ATX3; JOS; MJD; |
| | MJD1; SCA3 |
| AURKA | AIK; ARK1; AURA; AURORA2; |
| | BTAK; PPP1R47; STK15; |
| | STK6; STK7 |
| B3GALNT1 | B3GALT3; beta3Gal-T3; galT3; |
| | Gb4Cer; GLCT3; |
| | GLOB; P; P1 |
| B3GNTL1 | 3-Gn-T8; B3GNT8; beta-1; |
| | beta3Gn-T8; beta3GnTL1; |
| | BGnT-8 |
| B4GALT3 | beta4Gal-T3 |
| BAAT | BACAT; BAT |
| BAG1 | BAG-1; HAP; RAP46 |
| BAIAP2 | BAP2; FLAF3; IRSP53 |
| BAIAP2L2 | |
| BAZ2A | TIP5; WALp3 |
| BBX | ARTC1; HBP2; HSPC339; MDS001 |
| BCAR1 | CAS; CAS1; CASS1; CRKAS; |
| | P130Cas |
| BCL2 | Bcl-2; PPP1R50 |
| BCS1L | BCS; BCS1; BJS; FLNMS; |
| | GRACILE; h-BCS; h-BCS1; |
| | Hs.6719; MC3DN1; PTD |
| BET1 | HBET1 |
| BID | FP497 |
| BIRC2 | API1; c-IAP1; cIAP1; Hiap-2; |
| | HIAP2; MIHB; RNF48 |
| BPGM | DPGM |
| BPIFA2 | bA49G10.1; C20orf70; PSP; |
| | SPLUNC2 |
| BRINP2 | DBCCR1L2; FAM5B |
| BSG | 5F7; CD147; EMMPRIN; OK; TCSF |
| BTN3A2 | BT3.2; BTF4; BTN3.2; CD277 |
| C4BPB | C4BP |
| CACNA1A | EIEE42; FHM; HPCA; BI; |
| | CAV2.1; EA2; MHP1; MHP; |
| | CACNL1A4; SCA6CACNA1A; |
| | APCA |
| CALCOCO2 | NDP52 |
| CAPN11 | calpain11 |
| CASP12 | CASP-12; CASP12P1 |
| CASP8AP2 | CED-4; FLASH; RIP25 |
| CAV1 | BSCL3; CGL3; LCCNS; MSTP085; |
| | PPH3; VIP21 |
| CBX5 | HEL25; HP1; HP1A |
| CCDC120 | JM11 |
| CCDC17 | |
| CCDC186 | C10orf118 |
| CCDC51 | |
| CCN1 | CYR61; GIG1; IGFBP10 |
| CCND1 | BCL1; D11S287E; PRAD1; U21B31 |
| CCNT1 | CCNT; CYCT1; HIVE1 |
| CD2BP2 | FWP010; LIN1; PPP1R59; |
| | Snu40; U5-52K |
| CD9 | BTCC-1; DRAP-27; MIC3; MRP-1; |
| | TSPAN-29; TSPAN29 |
| CDC25C | CDC25; PPP1R60 |
| CDC42 | CDC42Hs; G25K; TKS |
| CDC7 | CDC7L1; HsCDC7; Hsk1; huCDC7 |
| CDCA7L | JPO2; R1; RAM2 |
| CDIP1 | C16orf5; CDIP; I1; LITAFL |
| CDK1 | CDC2; CDC28A; P34CDC2 |
| CDK11A | CDC2L2; CDC2L3; CDK11-p110; |
| | CDK11-p46; CDK11-p58; |
| | p58GTA; PITSLRE |
| CDKN1B | CDKN4; KIP1; MEN1B; MEN4; |
| | P27KIP1 |
| CEACAM7 | CGM2 |
| CEP295NL | DDC8; KIAA1731NL |
| CFLAR | c-FLIP; c-FLIPL; c-FLIPR; |
| | c-FLIPS; CASH; CASP8AP1; |
| | Casper; CLARP; FLAME; |
| | FLAME-1; FLAME1; FLIP; |
| | I-FLICE; MRIT |
| CHCHD7 | COX23 |
| CHIA | AMCASE; CHIT2; TSA1902 |
| CHIC1 | BRX |
| CHMP2A | BC-2; BC2; CHMP2; |
| | VPS2; VPS2A |
| CHRNA2 | |
| CLCN3 | ClC-3; CLC3 |
| CLEC12A | CD371; CLL-1; CLL1; |
| | DCAL-2; MICL |
| CLEC7A | BGR; CANDF4; CD369; |
| | CLECSF12; DECTIN1; |
| | SCARE2 |
| CLECL1 | DCAL-1; DCAL1 |
| CLRN1 | RP61; USH3; USH3A |
| CMSS1 | C3orf26 |
| CNIH1 | CNIH; CNIH-1; CNIL; TGAM77 |
| CNR1 | CANN6; CB-R; CB1; CB1A; |
| | CB1K5; CB1R; CNR |
| CNTN5 | HNB-2s; NB-2 |
| COG4 | CDG2J; COD1 |
| COMMD1 | C2orf5; MURR1 |
| COMMD5 | HCARG; HT002 |
| CPEB1 | CPE-BP1; CPEB; CPEB-1; |
| | h-CPEB; hCPEB-1 |
| CPS1 | CPSASE1; PHN |
| CRACR2B | EFCAB4A |
| CRBN | MRT2; MRT2A |
| CREM | CREM-2; hCREM-2; ICER |
| CRYBG1 | AIM1; ST4 |
| CSDE1 | D1S155E; UNR |
| CSF2RA | CD116; CDw116; CSF2R; CSF2RAX; |
| | CSF2RAY; CSF2RX; CSF2RY; |
| | GM-CSF-R-alpha; GMCSFR; |
| | GMR; SMDP4 |
| CSNK2A1 | CK2A1; CKII; CSNK2A3; OCNDS |
| CSTF3 | CSTF-77 |
| CTCFL | BORIS; CT27; CTCF-T; |
| | dJ579F20.2; HMGB1L1 |
| CTH | |
| CTNNA3 | ARVD13; VR22 |
| CTNNB1 | armadillo; CTNNB; MRD19 |
| CTNND1 | CAS; CTNND; p120; p120(CAS); |
| | p120(CTN); P120CAS; P120CTN |
| CTSL | MEPcathepsin L; CATL; CTSL1 |
| CUTA | ACHAP; C6orf82 |
| CXCR5 | BLR1; CD185; MDR15 |
| CYB5R3 | B5R; DIA1 |
| CYP24A1 | CP24; CYP24; HCAI; HCINF1; |
| | P450-CC24 |
| CYP3A5 | CP35; CYPIIIA5; P450PCN3; PCN3 |
| DAG1 | 156DAG; A3a; AGRNR; DAG; |
| | MDDGA9; MDDGC7; MDDGC9 |
| DAP3 | bMRP-10; DAP-3; MRP-S29; |
| | MRPS29 |
| DAXX | BING2; DAP6; EAP1 |
| DCAF4 | WDR21; WDR21A |
| DCAF7 | AN11; HAN11; SWAN-1; WDR68 |
| DCLRE1A | PSO2; SNM1; SNM1A |
| DCP1A | HSA275986; Nbla00360; |
| | SMAD4IP1; SMIF |
| DCTN1 | DAP-150; DP-150; P135 |
| DCTN2 | DCTN50; DYNAMITIN; |
| | HEL-S-77; RBP50 |
| DDX19B | DBP5; DDX19; RNAh |
| DDX46 | Prp5; PRPF5 |
| DEFB123 | DEFB-23; DEFB23; ESC42-RELD |
| DGKA | DAGK; DAGK1; DGK-alpha |
| DGKD | dgkd-2; DGKdelta |
| DHRS4 | CR; NRDR; PHCR; PSCD; |
| | SCAD-SRL; SDR-SRL; |
| | SDR25C1; SDR25C2 |
| DHX15 | DBP1; DDX15; HRH2; PRP43; |
| | PRPF43; PrPp43p |
| DIO3 | 5DIII; D3; DIOIII; TXDI3 |
| DLG1 | dJ1061C18.1.1; DLGH1; hdlg; |
| | SAP-97; SAP97 |
| DLL4 | hdelta2DLL4; AOS6 |
| DMD | BMD; CMD3B; DXS142; DXS164; |
| | DXS206; DXS230; DXS239; |
| | DXS268; DXS269; DXS270; |
| | DXS272; MRX85 |
| DMKN | UNQ729; ZD52F10 |
| DNAH6 | Dnahc6; DNHL1; HL-2; HL2 |
| DNAL4 | MRMV3; PIG27 |
| DUSP13 | BEDP; DUSP13A; DUSP13B; |
| | MDSP; SKRP4; TMDP |
| DUSP19 | DUSP17; LMWDSP3; SKRP1; |
| | TS-DSP1 |
| DYNC1I2 | DIC74; DNCI2; IC2 |
| DYNLRB2 | DNCL2B; DNLC2B; ROBLD2 |
| DYRK1A | DYRK; DYRK1; HP86; MNB; |
| | MNBH; MRD7 |
| ECI2 | ACBD2; dJ1013A10.3; DRS-1; |
| | DRS1; HCA88; PECI |
| ECT2 | ARHGEF31 |
| EIF1AD | haponin |
| EIF2B4 | EIF-2B; EIF2B; EIF2Bdelta |
| EIF4G1 | EIF-4G1; EIF4F; EIF4G; |
| | EIF4GI; P220; PARK18 |
| EIF4G2 | AAG1; DAP5; NAT1; P97 |
| EIF4G3 | eIF-4G 3; eIF4G 3; eIF4GII |
| ELANE | HLE; GE; NE; ELA2; HNE; |
| | SCN1ELANE; PMN-E |
| ELOVL6 | FACE; FAE; LCE |
| ELP5 | C17orf81; DERP6; HSPC002; |
| | MST071; MSTP071 |
| EMCN | EMCN2; MUC14 |
| ENO1 | ENO1L1; HEL-S-17; MPB1; |
| | NNE; PPH |
| EPB41 | 4.1R; EL1; HE |
| ERMN | JN; KIAA1189 |
| ERVV-1 | ENVV1; HERV-V1 |
| ESRRG | ERR3; ERRgamma; NR3B3 |
| ETFB | FP585; MADD |
| ETFBKMT | C12orf72; ETFB-KMT; METTL20 |
| ETV1 | ER81 |
| ETV4 | E1A-F; E1AF; PEA3; PEAS3 |
| EXD1 | EXDL1 |
| EXT1 | EXT; LGCR; LGS; TRPS2; TTV |
| EZH2 | ENX-1; ENX1; EZH1; EZH2b; |
| | KMT6; KMT6A; WVS; WVS2 |
| FAM111B | CANP; POIKTMP |
| FAM157A | |
| FAM213A | Adrx; C10orf58; PAMM |
| FBXO25 | FBX25 |
| FBXO9 | dJ341E18.2; FBX9; |
| | NY-REN-57; VCIA1 |
| FBXW7 | AGO; CDC4; FBW6; FBW7; |
| | FBX30; FBXO30; FBXW6; |
| | hAgo; hCdc4; SEL-10; |
| | SEL10 |
| FCMR | FAIM3; TOSO |
| FGF-9 | |
| FGF1 | AFGF; ECGF; ECGF-beta; |
| | ECGFA; ECGFB; FGF-1; |
| | FGF-alpha; FGFA; |
| | GLIO703; HBGF-1; |
| | HBGF1 |
| FGF2 | BFGF; FGF-2; FGFB; |
| | HBGF-2 |
| FHL5 | 1700027G07Rik; ACT; |
| | dJ393D12.2; FHL-5 |
| FMR1 | FMRP; FRAXA; POF; POF1; |
| | POFX |
| FN1 | CIG; ED-B; FINC; FN; |
| | FNZ; GFND; GFND2; |
| | LETS; MSF |
| FOXP1 | 12CC4; hFKH1B; HSPC215; |
| | MFH; QRF1 |
| FTH1 | HFE5; PLIFH-ferritin; |
| | PIG15; FTHL6; FHC; FTH |
| FUBP1 | FBP; FUBP; hDH V |
| G3BP1 | G3BP; HDH-VIII |
| GABBR1 | GABABR1; GABBR1-3; GB1; |
| | GPRC3A |
| GALC | |
| GART | AIRS; GARS; GARTF; |
| | PAIS; PGFT; PRGS |
| GAS7 | |
| gastrin | |
| GATA1 | ERYF1; GATA-1; GF-1; |
| | GF1; NF-E1; NFE1; |
| | XLANP; XLTDA; XLTT |
| GATA4 | MGC126629GATA-4 |
| GFM2 | EF-G2mt; EFG2; hEFG2; |
| | mEF-G 2; MRRF2; |
| | MST027; MSTP027; |
| | RRF2; RRF2mt |
| GHR | GHBP; GHIP |
| GJB2 | NSRD1; DFNB1; DFNB1A; |
| | CX26; DFNA3; DFNA3A; |
| | KID; HID; PPKCx26 |
| GLI1 | GLI |
| GLRA2 | GLR |
| GMNN | Gem; MGORS6 |
| GPAT3 | AGPAT 10; AGPAT10; |
| | AGPAT8; AGPAT9; |
| | HMFN0839; |
| | LPAAT-theta; MAG1 |
| GPATCH3 | GPATC3 |
| GPR137 | C11orf4; GPR137A; |
| | TM7SF1L1 |
| GPR34 | LYPSR1 |
| GPR55 | LPIR1 |
| GPR89A | GPHR; GPR89; GPR89B; |
| | SH120; UNQ192 |
| GPRASP1 | GASP; GASP-1; GASP1 |
| GRAP2 | GADS; GRAP-2; GRB2L; |
| | GRBLG; GrbX; Grf40; |
| | GRID; GRPL; Mona; P38 |
| GSDMB | GSDML; PP4052; PRO2521 |
| GSTO2 | bA127L20.1; GSTO 2-2 |
| GTF2B | TF2B; TFIIB |
| GTF2H4 | P52; TFB2; TFIIH |
| GUCY1B2 | |
| HAX1 | HCLSBP1; HS1BP1; SCN3 |
| HCST | DAP10; KAP10; PIK3AP |
| HIGD1A | HIG1; RCF1a |
| HIGD1B | CLST11240; CLST11240-15 |
| HIPK1 | Myak; Nbak2 |
| HIST1H1C | H1.2; H1C; H1F2; H1s-1 |
| HIST1H3H | H3/k; H3F1K; H3FK |
| HK1 | hexokinase; HK; HK1-ta; |
| | HK1-tb; HK1-tc; HKD; |
| | HKI; HMSNR; HXK1 |
| HLA-DRB4 | DR4; DRB4; HLA-DR4B |
| HMBS | PBG-D; PBGD; PORC; UPS |
| HMGA1 | HMG-R; HMGA1A; HMGIY |
| HNRNPC | C1; C2; HNRNP; HNRPC; SNRPC |
| HOPX | CAMEO; HOD; HOP; LAGY; |
| | NECC1; OB1; SMAP31; |
| | TOTO |
| HOXA2 | HOX1K; MCOHI |
| HOXA3 | HOX1; HOX1E |
| HPCAL1 | BDR1; HLP2; VILIP-3 |
| HR | ALUNC; AU; HSA277165; |
| | HYPT4; MUHH; MUHH1 |
| HSP90AB1 | D6S182; HSP84; HSP90B; |
| | HSPC2; HSPCB |
| HSPA1A | HEL-S-103; HSP70-1; |
| | HSP70-1A; HSP70.1; |
| | HSP70I; HSP72; |
| | HSPA1 |
| HSPA4L | APG-1; HSPH3; Osp94 |
| HSPA5 | BIP; GRP78; HEL-S-89n; |
| | MIF2 |
| HYPK | C15orf63; HSPC136 |
| IFFO1 | HOM-TES-103; IFFO |
| IFT74 | BBS20; CCDC2; CMG-1; CMG1 |
| IFT81 | CDV-1; CDV-1R; CDV1; |
| | CDV1R; DV1 |
| IGF1 | IGF-I; IGFI; MGF |
| IGF1R | CD221; IGFIR; IGFR; JTK13 |
| IGF2 | C11orf43; GRDF; IGF-II; |
| | PP9974 |
| IL11 | AGIF; IL-11 |
| IL17RE | |
| IL1RL1 | DER4; FIT-1; IL33R; ST2; |
| | ST2L; ST2V; T1 |
| IL1RN | DIRA; ICIL-1RA; IL-1ra; |
| | IL-1ra3; IL-1RN; IL1F3; |
| | IL1RA; IRAP; MVCD4 |
| IL32 | IL-32alpha; IL-32beta; |
| | IL-32delta; IL-32gamma; |
| | NK4; TAIF; TAIFa; |
| | TAIFb; TAIFc; TAIFd |
| IL6 | BSF-2; BSF2; CDF; HGF; |
| | HSF; IFN-beta-2; |
| | IFNB2; IL-6 |
| ILF2 | NF45; PRO3063 |
| ILVBL | 209L8; AHAS; HACL1L; |
| | ILV2H |
| INSR | CD220; HHF5 |
| INTS13 | ASUN; C12orf11; GCT1; |
| | Mat89Bb; NET48; |
| | SPATA30 |
| IP6K1 | IHPK1; PiUS |
| ITGA4 | CD49D; IA4 |
| ITGAE | CD103; HUMINAE |
| KCNE4 | MIRP3 |
| KERA | CNA2; KTN; SLRR2B |
| KIAA1324 | EIG121 |
| KIF2C | CT139; KNSL6; MCAK |
| KIZ | C20orf19; HT013; Kizuna; |
| | NCRNA00153; PLK1S1; |
| | RP69 |
| KLHL31 | bA345L23.2; BKLHD6; |
| | KBTBD1; KLHL |
| KLK7 | hK7; PRSS6; SCCE |
| KRR1 | HRB2; RIP-1 |
| KRT14 | CK14; EBS3; EBS4; |
| | K14; NFJ |
| KRT17 | 39.1; CK-17; K17; PC; |
| | PC2; PCHC1 |
| KRT33A | Ha-3I; HA3I; hHa3-I; K33A; |
| | Krt1-3; KRTHA3A |
| KRT6A | CK-6C; CK-6E; CK6A; CK6C; |
| | CK6D; K6A; K6C; K6D; |
| | KRT6C; KRT6D; PC3 |
| KRTAP10-2 | KAP10.2; KAP18-2; KAP18.2; |
| | KRTAP10.2; KRTAP18-2; |
| | KRTAP18.2 |
| KRTAP13-3 | KAP13.3 |
| KRTAP13-4 | KAP13.4 |
| KRTAP5-11 | KRTAP5-5; KRTAP5-6; |
| | KRTAP5.11 |
| KRTCAP2 | KCP2 |
| LACRT | |
| LAMB1 | CLM; LIS5 |
| LAMB3 | AI1A; BM600-125KDA; |
| | LAM5; LAMNB1 |
| LANCL1 | GPR69A; p40 |
| LBX2 | LP3727 |
| LCAT | |
| LDHA | GSD11; HEL-S-133P; LDHM; |
| | PIG19 |
| LDHAL6A | LDH6A |
| LEF1 | LEF-1; TCF10; TCF1ALPHA; |
| | TCF7L3 |
| LINC-PINT | LincRNA-Pint; MKLN1-AS1; |
| | PINT |
| LMO3 | RBTN3; RBTNL2; Rhom-3; |
| | RHOM3 |
| LRRC4C | NGL-1;NGL1 |
| LRRC7 | DENSIN |
| LRTOMT | CFAP111; DFNB63; LRRC51 |
| LSM5 | YER146W |
| LTB4R | BLT1; BLTR; CMKRL1; GPR16; |
| | LTB4R1; LTBR1; P2RY7; |
| | P2Y7 |
| LYRM1 | A211C6.1 |
| LYRM2 | DJ122O8.2 |
| MAGEA11 | CT1.11; MAGE-11; MAGE11; |
| | MAGEA-11 |
| MAGEA8 | CT1.8; MAGE8 |
| MAGEB1 | CT3.1; DAM10; MAGE-Xp; |
| | MAGEL1 |
| MAGEB16 | |
| MAGEB3 | CT3.5 |
| MAPT | DDPAC; FTDP-17; MAPTL; |
| | MSTD; MTBT1; MTBT2; |
| | PPND; PPP1R103; TAU |
| MARS | CMT2U; ILFS2; ILLD; METRS; |
| | MRS; MTRNS; SPG70 |
| MC1R | CMM5; MSH-R; SHEP2 |
| MCCC1 | MCC-B; MCCA |
| METTL12 | U99HG |
| METTL7A | AAM-B |
| MIA2 | CTAGE5; MEA6; MGEA; MGEA11; |
| | MGEA6 |
| MITF | bHLHe32; CMM8; COMMAD; MI; |
| | WS2; WS2A |
| MKLN1 | TWA2 |
| MNT | bHLHd3; MAD6; MXD6; ROX |
| MORF4L2 | MORFL2; MRGX |
| MPD6 | |
| MRFAP1 | PAM14; PGR1 |
| MRPL21 | L21mt; MRP-L21 |
| MRPS12 | MPR-S12; MT-RPS12; RPMS12; |
| | RPS12; RPSM12 |
| MSI2 | MSI2H |
| MSLN | MPF; SMRP |
| MSN | HEL70; IMD50 |
| MT2A | MT2 |
| MTFR1L | FAM54B; HYST1888; MST116; |
| | MSTP116 |
| MTMR2 | CMT4B; CMT4B1 |
| MTRR | cblE; MSR |
| MTUS1 | ATBP; ATIP; ATIP3; ICIS; |
| | MP44; MTSG1 |
| MYB | c-myb; c-myb_CDS; Cmyb; efg |
| MYC | bHLHe39; c-Myc; MRTL; MYCC |
| MYCL | bHLHe38; L-Myc; LMYC; MYCL1 |
| MYCN | bHLHe37; MODED; N-myc; |
| | NMYC; ODED |
| MYL10 | MYLC2PL; PLRLC |
| MYL3 | CMH8; MLC-1V/sb; MLC1SB; |
| | MLC1V; VLC1; VLC1 |
| MYLK | AAT7; KRP; MLCK; MLCK1; |
| | MLCK108; MLCK210; |
| | MSTP083; MYLK1; smMLCK |
| MYO1A | BBMI; DFNA48; MIHC; MYHL |
| MYT2 | |
| MZB1 | MEDA-7; PACAP; pERp1 |
| NAP1L1 | NAP1; NAP1L; NRP |
| NAV1 | POMFIL3; STEERIN1; |
| | UNC53H1 |
| NBAS | ILFS2; NAG; SOPH |
| NCF2 | NCF-2; NOXA2; P67-PHOX; |
| | P67PHOX |
| NDRG1 | CAP43; CMT4D; DRG-1; DRG1; |
| | GC4; HMSNL; NDR1; NMSL; |
| | PROXY1; RIT42; RTP; |
| | TARG1; TDD5 |
| NDST2 | HSST2; NST2 |
| NDUFA7 | B14.5a; CI-B14.5a |
| NDUFB11 | CI-ESSS; ESSS; Np15; |
| | NP17.3; P17.3 |
| NDUFC1 | KFYI |
| NDUFS1 | CI-75k; CI-75Kd; PRO1304 |
| NEDD4L | hNEDD4-2; NEDD4-2; |
| | NEDD4.2; PVNH7; RSP5 |
| NFAT5 | NF-AT5; NFATL1; NFATZ; |
| | OREBP; TONEBP |
| NFE2L2 | HEBP1; NRF2 |
| NFIA | CTF; NF-I/A; NF1-A; |
| | NFI-A; NFI-L |
| NHEJ1 | XLF |
| NHP2 | DKCB2; NHP2P; NOLA2 |
| NIT1 | |
| NKRF | ITBA4; NRF |
| NME1-NME2 | NM23-LV; NMELV |
| NPAT | E14; E14/NPAT; p220 |
| NR3C1 | GCCR; GCR; GCRST; GR; |
| | GRL |
| NRBF2 | COPR; COPR1; COPR2; |
| | NRBF-2 |
| NRF1 | ALPHA-PAL |
| NTRK2 | EIEE58; GP145-TrkB; |
| | OBHD; trk-B; TRKB |
| NUDCD1 | CML66; OVA66 |
| NXF2 | CT39; TAPL-2; TCP11X2 |
| NXT2 | P15-2 |
| ODC1 | ODC |
| ODF2 | CT134; ODF2/1; ODF2/2; |
| | ODF84 |
| OPTN | ALS12; FIP2; GLC1E; |
| | HIP7; HYPL; NRP; |
| | TFIIIA-INTP |
| OR10R2 | OR1-8; OR10R2Q |
| OR11L1 | |
| OR2M2 | OR2M2Q; OST423 |
| OR2M3 | OR1-54; OR2M3P; OR2M6; |
| | OST003 |
| OR2M5 | OR2M5P |
| OR2T10 | OR1-64 |
| OR4C15 | OR11-127; OR11-134 |
| OR4F17 | OR4F11P; OR4F18; OR4F19 |
| OR4F5 | |
| OR5H1 | HSHTPCRX14; HTPCRX14 |
| OR5K1 | HSHTPCRX10; HTPCRX10; |
| | OR3-8 |
| OR6C3 | OST709 |
| OR6C75 | |
| OR6N1 | OR1-22 |
| OR7G2 | OR19-6; OST260 |
| P2RY4 | NRU; P2P; P2Y4; UNR |
| PAN2 | USP52 |
| PAQR6 | PRdelta |
| PARP4 | ADPRTL1; ARTD4; p193; |
| | PARP-4; PARPL; PH5P; |
| | VAULT3; VPARP; VWA5C |
| PARP9 | ARTD9; BAL; BAL1; |
| | MGC:7868 |
| PC | PCB |
| PCBP4 | CBP; LIP4; MCG10 |
| PCDHGC3 | PC43; PCDH-GAMMA-C3; |
| | PCDH2 |
| PCLAF | KIAA0101; L5; NS5ATP9; |
| | OEATC; OEATC-1; OEATC1; |
| | p15(PAF); p15/PAF; |
| | p15PAF; PAF; PAF15 |
| PDGFB | c-sis; IBGC5; PDGF-2; |
| | PDGF2; SIS; SSV |
| PDZRN4 | LNX4; SAMCAP3L |
| PELO | CGI-17; PRO1770 |
| PEMT | PEAMT; PEMPT; PEMT2; |
| | PLMT; PNMT |
| PEX2 | PAF1; PBD5A; PBD5B; PMP3; |
| | PMP35; PXMP3; RNF72; ZWS3 |
| PFKM | ATP-PFK; GSD7; PFK-1; PFK1; |
| | PFKA; PFKX; PPP1R122 |
| PGBD4 | NA |
| PGLYRP3 | PGLYRPIalpha; PGRP-Ialpha; |
| | PGRPIA |
| PHLDA2 | BRW1C; BWR1C; HLDA2; IPL; |
| | TSSC3 |
| PHTF1 | PHTF |
| PI4KB | NPIK; PI4K-BETA; PI4K92; |
| | PI4KBETA; PI4KIIIBETA; |
| | PIK4CB |
| PIGC | GPI2 |
| PKD2L1 | PCL; PKD2L; PKDL; TRPP3 |
| PKM | CTHBP; HEL-S-30; OIP3; |
| | PK3; PKM2; TCB; THBP1 |
| PLCB4 | ARCND2; PI-PLC |
| PLD3 | AD19; HU-K4; HUK4 |
| PLEKHA1 | TAPP1 |
| PLEKHB1 | KPL1; PHR1; PHRET1 |
| PLS3 | BMND18; T-plastin |
| PML | RNF71; TRIM19PML; PP8675; |
| | MYL |
| PNMA5 | |
| PNN | DRS; DRSP; memA; SDK3 |
| POC1A | PIX2; SOFT; WDR51A |
| POC1B | CORD20; PIX1; TUWD12; |
| | WDR51B |
| POLD2 | |
| POLD4 | p12; POLDS |
| POU5F1 | Oct-3; Oct-4; OCT3; OCT4; |
| | OTF-3; OTF3; OTF4 |
| PPIG | CARS-Cyp; CYP; SCAF10; SRCyp |
| PQBP1 | MRX2; MRX55; MRXS3; MRXS8; |
| | NPW38; RENS1; SHS |
| PRAME | CT130; MAPE; OIP-4; OIP4 |
| PRPF4 | HPRP4; HPRP4P; PRP4; Prp4p; |
| | RP70; SNRNP60 |
| PRR11 | NA |
| PRRT1 | C6orf31; DSPD1; IFITMD7; NG5 |
| PRSS8 | CAP1; PROSTASIN |
| PSMA2 | HC3; MU; PMSA2; PSC2 |
| PSMA3 | HC8; PSC3 |
| PSMA4 | HC9; HsT17706; PSC9 |
| PSMD11 | p44.5; Rpn6; S9 |
| PSMD4 | AF; AF-1; ASF; MCB1; |
| | pUB-R5; Rpn10; S5A |
| PSMD6 | p42A; p44S10; Rpn7; S10; |
| | SGA-113M |
| PSME3 | HEL-S-283; Ki; PA28-gamma; |
| | PA28G; PA28gamma; |
| | REG-GAMMA |
| PSMG3 | C7orf48; PAC3 |
| PTBP3 | ROD1 |
| PTCH1 | NBCCS; PTC; PTC1; PTCH11PTCH1b; |
| | HPE7; PTCH; BCNS |
| PTHLH | BDE2; HHM; PLP; PTHR; PTHRP |
| PTPRD | HPTP; HPTPD; HPTPDELTA; |
| | PTPD; RPTPDELTA |
| PUS7L | |
| PVRIG | C7orf15; CD112R |
| QPRT | HEL-S-90n; QPRTase |
| RAB27A | GS2; HsT18676; RAB27; RAM |
| RAB7B | RAB7 |
| RABGGTB | GGTB |
| RAET1E | bA350J20.7; LETAL; N2DL-4; |
| | NKG2DL4; RAET1E2; RL-4; |
| | ULBP4 |
| RALGDS | RalGEF; RGDS; RGF |
| RALYL | HNRPCL3 |
| RARB | HAP; MCOPS12; NR1B2; |
| | RARbeta1; RRB2 |
| RCVRN | RCV1 |
| REG3G | LPPM429; PAP IB; PAP-1B; |
| | PAP1B; PAPIB; REG III; |
| | REG-III; UNQ429 |
| RFC5 | RFC36 |
| RGL4 | Rgr |
| RGS19 | GAIP; RGSGAIP |
| RGS3 | C2PA; RGP3 |
| RHD | CD240D; DIIIc; RH; RH30; |
| | Rh4; RHCED; RhDCw; |
| | RHDel; RHDVA(TT); RhII; |
| | RhK562-II; RhPI; RHPII; |
| | RHXIII |
| RINL | |
| RIPOR2 | C6orf32; DFNB104; DIFF40; |
| | DIFF48; FAM65B; MYONAP; |
| | PL48 |
| RITA1 | C12orf52; RITA |
| RMDN2 | BLOCK18; FAM82A; FAM82A1; |
| | PRO34163; PYST9371; |
| | RMD-2; RMD2; RMD4 |
| RNASE1 | RAC1; RIB1; RNS1 |
| RNASE4 | RAB1; RNS4 |
| RNF4 | RES4-26; SLX5; SNURF |
| RPA2 | RP-A p32; RPA32RPA2; |
| | REPA2; RP-A p34 |
| RPL17 | L17; PD-1; RPL23 |
| RPL21 | HYPT12; L21 |
| RPL26L1 | RPL26P1 |
| RPL28 | L28 |
| RPL29 | HIP; HUMRPL29; L29; |
| | RPL29P10; RPL29_3_370 |
| RPL41 | L41 |
| RPL9 | L9; NPC-A-16 |
| RPS11 | S11 |
| RPS13 | S13 |
| RPS14 | EMTB; S14 |
| RRBP1 | ES/130; ES130; hES; RRp |
| RSU1 | RSP-1 |
| RTP2 | Z3CXXC2 |
| RUNX1 | AML1; AML1-EVI-1; AMLCR1; |
| | CBF2alpha; CBFA2; EVI-1; |
| | PEBP2aB; PEBP2alpha |
| RUNX1T1 | AML1-MTG8; AML1T1; CBFA2T1; |
| | CDR; ETO; MTG8; ZMYND2 |
| RUNX2 | CLCD; AML3; OSF2; CBF-alpha-1; |
| | CBFA1; CCD; PEBP2aARNUX2; |
| | OSF-2; PEA2aA; CCD1 |
| RUSC1 | NESCA |
| RXRG | NR2B3; RXRC |
| S100A13 | |
| S100A4 | 18A2; 42A; CAPL; FSP1; |
| | MTS1; P9KA; PEL98 |
| SAT1 | DC21; KFSD; KFSDX; SAT; |
| | SSAT; SSAT-1 |
| SCHIP1 | SCHIP-1 |
| SCMH1 | Scml3 |
| SEC14L1 | PRELID4A; SEC14L |
| SEMA4A | CORD10; RP35; SEMAB; SEMB |
| SERPINA1 | A1A; A1AT; AAT; alpha1AT; |
| | PI; PI1; PRO2275 |
| SERPINB4 | LEUPIN; PI11; SCCA-2; |
| | SCCA1; SCCA2 |
| SERTAD3 | RBT1 |
| SFTPD | COLEC7; PSP-D; SFTP4; |
| | SP-D |
| SH3D19 | EBP; Eve-1; EVE1; Kryn; |
| | SH3P19 |
| SHC1 | SHC; SHCA |
| SHMT1 | CSHMT; SHMT |
| SHPRH | bA545I5.2 |
| SIM1 | bHLHe14 |
| SIRT5 | SIR2L5 |
| SLC11A2 | AHMIO1; DCT1; DMT1; NRAMP2 |
| SLC12A4 | CTC-479C5.17; hKCC1; KCC1 |
| SLC16A1 | HHF7; MCT; MCT1; MCT1D |
| SLC25A3 | OK/SW-cl.48; PHC; PTP |
| SLC26A9 | |
| SLC5A11 | KST1; RKST1; SGLT6; SMIT2 |
| SLC6A12 | BGT-1; BGT1; GAT2 |
| SLC6A19 | B0AT1; HND |
| SLC7A1 | ERR; CAT-1; ATRC1; |
| | HCAT1; REC1LCAT-1 |
| SLFN11 | SLFN8/9 |
| SLIRP | C14orf156; DC50; PD04872 |
| SMAD5 | DWFC; JV5-1; MADH5 |
| SMARCAD1 | ADERM; BASNS; ETL1; HEL1 |
| SNCA | PARK4; PARK1; NACP; PD1SNCA |
| SNRNP200 | ASCC3L1; BRR2; HELIC2; |
| | RP33; U5-200KD |
| SNRPB2 | Msl1; U2B″ |
| SNX12 | |
| SOD1 | ALS; ALS1; HEL-S-44; |
| | homodimer; hSod1; |
| | IPOA; SOD |
| SOX13 | ICA12; Sox-13 |
| SOX5 | L-SOX5; L-SOX5B; L-SOX5F; |
| | LAMSHF |
| SP8 | BTD |
| SPARCL1 | MAST 9; MAST9; PIG33; SC1 |
| SPATA12 | SRG5 |
| SPATA31C2 | FAM75C2 |
| SPN | CD43; GALGP; GPL115; LSN |
| SPOP | BTBD32; TEF2 |
| SQSTM1 | A170; DMRV; FTDALS3; NADGP; |
| | OSIL; p60; p62; p62B; |
| | PDB3; ZIP3 |
| SRBD1 | |
| SRC | SRC1; c-SRC; ASV; |
| | THC6c-Src; p60-Src |
| SREBF1 | SREBP-1c; bHLHd1; |
| | SREBP1SREBP-1 |
| SRPK2 | SFRSK2 |
| SSB | La; La/SSB; LARP3 |
| SSBP1 | Mt-SSB; mtSSB; SOSS-B1; SSBP |
| ST3GAL6 | SIAT10; ST3GALVI |
| STAB1 | CLEVER-1; FEEL-1; FELE-1; |
| | FEX1; SCARH2; STAB-1 |
| STAMBP | AMSH; MICCAP |
| STAU1 | Stau1; STAUStau1; Stau2; |
| | PPP1R150; Stau3 |
| STK16 | hPSK; KRCT; MPSK; PKL12; |
| | PSK; TSF1 |
| STK24 | HEL-S-95; MST3; MST3B; |
| | STE20; STK3 |
| STK38 | NDR; NDR1 |
| STMN1 | C1orf215; Lag; LAP18; OP18; |
| | PP17; PP19; PR22; SMN |
| STX7 | |
| SULT2B1 | HSST2 |
| SYK | p72-Syk |
| SYNPR | SPO |
| TAF1C | MGC:39976; SL1; TAFI110; |
| | TAFI95 |
| TAGLN | SM22; SMCC; TAGLN1; |
| | WS3-10 |
| TANK | I-TRAF; ITRAF; TRAF2 |
| TAS2R40 | GPR60; T2R40; T2R58 |
| TBC1D15 | RAB7-GAP |
| TBXAS1 | BDPLT14; CYP5; CYP5A1; |
| | GHOSAL; THAS; TS; |
| | TXAS; TXS |
| TCF4 | bHLHb19; E2-2; FECD3; |
| | ITF-2; ITF2; PTHS; |
| | SEF-2; SEF2; SEF2-1; |
| | SEF2-1A; SEF2-1B; |
| | SEF2-1D; TCF-4 |
| TDGF1 | CR; CRGF; CRIPTO |
| TDP2 | ARC34; p34-Arc; PNAS-139; |
| | PRO2446 |
| TDRD3 | |
| TDRD5 | TUDOR3 |
| TESK2 | |
| THAP6 | |
| THBD | TMTM; THRM; AHUS6; BDCA3; |
| | CD141; THPH12 |
| THTPA | THTP; THTPASE |
| TIAM2 | STEF; TIAM-2 |
| TKFC | DAK; NET45 |
| TKTL1 | TKR; TKT2 |
| TLR10 | CD290 |
| TM9SF2 | P76 |
| TMC6 | EV1; EVER1; EVIN1; LAK-4P |
| TMCO2 | dJ39G22.2 |
| TMED10 | p23; P24(DELTA); p24d1; |
| | S31I125; S31III125; |
| | Tmp-21-I; TMP21 |
| TMEM116 | |
| TMEM126A | OPA7 |
| TMEM159 | |
| TMEM208 | HSPC171 |
| TMEM230 | C20orf30; dJ1116H23.2.1; |
| | HSPC274 |
| TMEM67 | JBTS6; MECKELIN; MKS3; |
| | NPHP11; TNEM67 |
| TMPRSS13 | MSP; MSPL; MSPS; TMPRSS11 |
| TMUB2 | FP2653 |
| TNFSF4 | CD134L; CD252; GP34; OX-40L; |
| | OX4OL; TNLG2B; TXGP1 |
| TNIP3 | ABIN-3; LIND |
| TP53 | BCC7; LFS1; P53; TRP53 |
| TP73 | P73p73 |
| TRAF1 | EBI6; MGC:10353 |
| TRAK1 | MILT1; OIP106 |
| TRIM31 | C6orf13; HCG1; HCGI; RNF |
| TRIM6 | RNF89 |
| TRMT1 | TRM1 |
| TRMT2B | CXorf34; dJ341D10.3 |
| TRPM7 | ALSPDC; CHAK; CHAK1; |
| | LTrpC-7; LTRPC7; |
| | TRP-PLIK |
| TRPM8 | LTRPC6; TRPP8 |
| TSPEAR | C21orf29; DFNB98; TSP-EAR |
| TTC39B | C9orf52 |
| TTLL11 | bA244O19.1; C9orf20 |
| TUBB6 | HsT1601; TUBB-5 |
| TXLNB | C6orf198; dJ522B19.2; |
| | LST001; MDP77 |
| TXNIP | ARRDC6; EST01027; HHCPA78; |
| | THIF; VDUP1 |
| TXNL1 | HEL-S-114; TRP32; Txl; |
| | TXL-1; TXNL |
| TXNRD1 | GRIM-12; TR; TR1; TRXR1; |
| | TXNR |
| TYROBP | DAP12; KARAP; PLOSL |
| U2AF1 | FP793; RN; RNU2AF1; |
| | U2AF35; U2AFBP |
| UBA1 | A1S9; A1S9T; A1ST; AMCX1; |
| | CFAP124; GXP1; POC20; |
| | SMAX2; UBA1A; UBE1; |
| | UBE1X |
| UBE2D3 | E2(17)KB3; UBC4/5; UBCH5C |
| UBE2I | C358B7.1; P18; UBC9 |
| UBE2L3 | E2-F1; L-UBC; UBCH7; UbcM4 |
| UBE2V1 | CIR1; CROC-1; CROC1; UBE2V; |
| | UEV-1; UEV1; UEV1A |
| UBE2V2 | DDVit-1; DDVIT1; EDAF-1; |
| | EDPF-1; EDPF1; MMS2; |
| | UEV-2; UEV2 |
| UMPS | OPRT |
| UNG | DGU; HIGM4; HIGM5; UDG; |
| | UNG1; UNG15; UNG2 |
| UPP2 | UDRPASE2; UP2; UPASE2 |
| USMG5 | bA792D24.4; DAPIT; |
| | HCVFTP2 |
| USP18 | PTORCH2; ISG43; |
| | UBP43USP18-sf |
| UTP14A | dJ537K23.3; NYCO16; SDCCAG16 |
| UTRN | DMDL; DRP; DRP1 |
| UTS2 | PRO1068; U-II; UCN2; UII |
| VDR | NR1I1; PPP1R163 |
| VEGFA | MVCD1; VEGF; VPF |
| VEPH1 | MELT; VEPH |
| VIPAS39 | C14orf133; hSPE-39; SPE-39; |
| | SPE39; VIPAR; VPS16B |
| VPS29 | DC15; DC7; PEP11 |
| VSIG10L | |
| WDHD1 | AND-1; AND1; CHTF4; CTF4 |
| WDR12 | YTM1 |
| WDR4 | TRM82; TRMT82 |
| WDR45 | JM5; NBIA4; NBIA5; WDRX1; |
| | WIPI-4; WIPI4 |
| WDYHV1 | C8orf32 |
| WRAP53 | DKCB3; TCAB1; WDR79 |
| XIAP | API3; BIRC4; hIAP-3; hIAP3; |
| | IAP-3; ILP1; MIHA; XLP2 |
| XPNPEP3 | APP3; ICP55; NPHPL1 |
| YAP1 | COB1; YAP; YAP2; YAP65; YKI |
| YWHAZ | 14-3-3-zeta; HEL-S-3; HEL-S-93; |
| | HEL4; KCIP-1; YWHAD |
| YY1AP1 | GRNG; HCCA1; HCCA2; YY1AP |
| ZBTB32 | FAXF; FAZF; Rog; TZFP; ZNF538 |
| ZNF146 | OZF |
| ZNF250 | ZFP647; ZNF647 |
| ZNF385A | HZF; RZF; ZFP385; ZNF385 |
| ZNF408 | EVR6; RP72 |
| ZNF410 | APA-1; APA1 |
| ZNF423 | Ebfaz; hOAZ; JBTS19; NPHP14; |
| | OAZ; Roaz; Zfp104; ZFP423 |
| ZNF43 | HTF6; KOX27; ZNF39L1 |
| ZNF502 | |
| ZNF512 | |
| ZNF513 | HMFT0656; RP58 |
| ZNF580 | |
| ZNF609 | |
| ZNF707 | |
| ZNRD1 | HTEX-6; HTEX6; hZR14; Rpal2; |
| | tctex-6; TCTEX6; TEX6; ZR14 |

TABLE 4
provides a list of four different plasmids transformed into E. coli
bacteria (FEC21) to encode an RNA molecule comprising an IRES
element, a luc coding sequence, and a poly-A tail; each
bacterial transformant was screened for the presence of the
associated IRES element by PCR.

| | | PCR |
|---|---|---|
| | | Product |
| Plasmid | 5′ IRES Element | (bp) |
| pSiVEC2_circCRPV-lucA | CrPV with bacteriophage | 104 |
| | T4 permuted-intron-exon | 216 |
| | sequence | |
| pSiVEC2_FMDV-lucA | FMDV (Foot-and-mouth | 219 |
| | disease virus) | |
| pSiVEC2_CSFV-lucA | CSFV (Classical swine | 281 |
| | fever virus) | |
| pSiVEC2_CRPV-lucA | CrPV (Cricket paralysis | 104 |
| | virus) | |

TABLE 5
provides a summary of PCR results of post-bacterial generated and delivered
eukaryote-translatable mRNA to A549 cells, confirming all components were present
in the A549 cells, including each of the IRES elements, the gene coding sequence
(luc), and the poly-A tail, verified by RT with oligo(dT) primers.

| | | | IRES | luc | Poly-A |
|---|---|---|---|---|---|
| Construct | Colony | Type | PCR | PCR | RT |
| FEC21/ | A | Bacteria only | + | + | + |
| pSiVEC2_circCRPV-lucA | | A549 + bacteria | + | + | + |
| | B | Bacteria only | + | + | + |
| | | A549 + bacteria | + | + | + |
| FEC21/ | A | Bacteria only | + | + | + |
| pSiVEC2_FMDV-lucA | | A549 + bacteria | + | + | + |
| | B | Bacteria only | + | + | + |
| | | A549 + bacteria | + | + | + |
| FEC21/ | A | Bacteria only | + | + | + |
| pSiVEC2_CSFV-lucA | | A549 + bacteria | + | + | + |
| | B | Bacteria only | + | + | + |
| | | A549 + bacteria | + | + | + |
| FEC21/ | A | Bacteria only | + | + | + |
| pSiVEC2_CRPV-lucA | | A549 + bacteria | + | + | + |
| | B | Bacteria only | + | + | + |
| | | A549 + bacteria | + | + | + |
| FEC21 (untransformed) | A | Bacteria only | −, −, − | − | − |
| Untreated A549 cells | n/a | A549 only | −, −, − | − | + |

TABLE 6
Selected IRES sequences

Cricket paralysis virus (CrPV)-IRES-[SEQ ID NO. 1]
5′-AAAATGTGATCTTGCTTGTAAATACAATTTTGAGAGGTTAATAAATTACAAGTAGT
GCTATTTTTGTATTTAGGTTAGCTATTTAGCTTTACGTTCCAGGATGCCTAGTGGCA
GCCCCACAATATCCAGGAAGCCCTCTCTGCGGTTTTTCAGATTAGGTAGTCGAAAA
ACCTAAGAAATTTACCTGCT-3′
Foot and mouth disease virus (FMDV)-IRES-[SEQ ID NO. 2]
5′-TGCAGGTAGCCCCAACTGACACAAACCGTGCAACTTGGAACCCCGCCTGGGCTTT
CCAGGTCTAGAGGGGTGACGCCTTGTACTGTGTTTGACTCCACGCTCGGTCCACTA
GCGAGTGTTAGTAGTAGTACTGTTGCTTCGTAGCGGAGCATGACGGCCGTGGGAA
TCCCTCCTTGGCAACAAGGACCCACGGGGCCGAAAGCCACGTCCTGAAGGACCCG
TCATGTGTGCAACCCCAGCACGGCAGCTTTATTATGAAACCCACTTTAAGGTGACA
CTGATACTGGTACTCAAACACTGGTGACAGGCTAAGGATGCCCTTCAGGTACCCC
GAGGTAACACGCGACACTCGGGATCTGAGAAGGGGACTGGGGCTTCTATAAAAGT
GCCCAGTTTAAAAAGCTTCTATGCCTGGATAGGCGACCGGAGGCCGGCGCCTTTC
CTTTGACCACTACTGTTTAC-3′
Classical swine fever virus (CSFV)-IRES- [SEQ ID NO. 3]
GTTAGCTCTTTCTCGTATACGATATTGGATACACTAAATTTCGATTTGCTCTAGGG
CACCOCTCCAGCGACGGCCGAAATGGGCTAGCCATGCCCATAGTAGGACTAGCAA
ACGGAGGGACTAGCCGTAGTGGCGAGCTCCCTGGGTGGTCTAAGTCCTGAGTACA
GGACAGTCGTCAGTAGTTCGACGTGAGCACTRGCCCACCTCGAGATGCTACGTGG
ACGAGGGCATGCCCAAGACACACCTTAACCCTGGCGGGGGTCGCTAGGGTGAAAT
CACATTATGTGATGGGGGTACGACCTGATAGGGTGCTGCAGAGGCCCACTAGCAG
GCTAGTATAAAAATCTCTGCTGTACATGGCAC
Circular CrPV with circularization components bolded
(spacer, T4 phage intron and exon elements)-[SEQ ID NO. 4]
5′-GGGAGACCCTCGAATGGAATTGGTTCTACATAAATGCCTAACGACTATCCCT
TTGGGGAGTAGGGTCAAGTGACTCGAAACGATAGACAACTTGCTTTAACAAG
TTGGAGATATAGTCTGCTCTGCATGGTGACATGCAGCTGGATATAATTCCGG
GGTAAGATTAACGACCTTATCTGAACATAATGCTACCGTTTAATATTGCGTCA
GGTAGTAAACTACTAACTACAACCTGCTGAAGCAAAAATGTGATCTTGCTTGTA
AATACAATTTTGAGAGGTTAATAAATTACAAGTAGTGCTATTTTTGTATTTAGGTT
AGCTATTTAGCTTTACGTTCCAGGATGCCTAGTGGCAGCCCCACAATATCCAGGAA
GCCCTCTCTGCGGTTTTTCAGATTAGGTAGTCGAAAAACCTAAGAAATTTACCTGC
TGGTAGTAAACTACTAACTACAACCTGCTGAAGCAGATGTTTTCTTGGGTTAA
TTGAGGCCTGAGTATAAGGTGACTTATACTTGTAATCTATCTAAACGGGGAA
CCTCTCTAGTAGACAATCCCGTGCTAAATTGTAGGACTAATTCCATTTATCAG
ATTTCTAG-3′
All sequences are listed 5′ to 3′

1. A system for generating eukaryote-translatable mRNA comprising a bacterium engineered to have at least one expression cassette encoding a eukaryote-translatable mRNA comprising a 5′ pseudo-cap element, a nucleic acid sequence encoding a polypeptide, and a poly-A tail, wherein transcription of the eukaryote-translatable mRNA is under the control of a prokaryotic promoter.

2. The system for generating eukaryote-translatable mRNA according to claim 1 wherein the 5′ pseudo-cap element is an internal ribosome entry sequence (IRES).

3. The system for generating eukaryote-translatable mRNA according to claim 1 wherein the IRES is an IRES selected from the group consisting of Cricket paralysis virus (CrPV) IRES, Foot and mouth disease virus (FMDV) IRES and Classical swine fever virus (CSFV) IRES or an IRES listed in tables 1-3.

4. The system for generating eukaryote-translatable mRNA according to claim 1 wherein the bacterium is a nonpathogenic bacterium engineered to have at least one invasion factor.

5. The system for generating eukaryote-translatable mRNA according to claim 1 wherein the bacterium is engineered to transcribe a eukaryote-translatable mRNA that is circularized in the bacteria upon its transcription.

6. (canceled)

7. A system for generating eukaryote-translatable mRNA comprising a bacterium having at least one expression cassette comprising a sequence encoding a eukaryote-translatable mRNA, wherein transcription of the sequence encoding the eukaryote-translatable mRNA is under the control of a promoter that is inactive in a eukaryotic cell and wherein the eukaryote-translatable mRNA molecule comprises eukaryote-derived sequence elements that allow translation of a polypeptide in a eukaryotic cell.

8. The system for generating eukaryote-translatable mRNA according to claim 7 wherein the sequence encoding the eukaryote-translatable mRNA is engineered to be on the chromosome of the bacterium.

9. The system for generating eukaryote-translatable mRNA according to claim 7 wherein the expression cassette is a plasmid comprising a sequence encoding at least one mRNA molecule containing eukaryote-translatable elements.

10. The system for generating eukaryote-translatable mRNA according to claim 7 wherein the expression cassette comprises a sequence encoding a eukaryote-translatable mRNA that has a 5′-end comprising a 5′ cap or pseudo cap-like element capable of eukaryotic ribosome recruitment and a 3′ end containing a poly-A tail resulting in a eukaryote-translatable mRNA molecule produced within the bacterial cell.

11. The system for generating eukaryote-translatable mRNA according to claim 7 wherein the eukaryote-translatable elements for translation into a protein comprises a viral or non-viral eukaryotic cellular internal ribosome entry site (IRES) element.

12. The system for generating eukaryote-translatable mRNA according to claim 11 wherein the viral or non-viral eukaryotic cellular internal ribosome entry site (IRES) element is selected from the group consisting of Cricket paralysis virus (CrPV) IRES, Foot and mouth disease virus (FMDV) IRES, Classical swine fever virus (CSFV) IRES, or an IRES listed in tables 1-3.

13. The system for generating eukaryote-translatable mRNA according to claim 7 wherein the sequence encoding a eukaryote-translatable mRNA includes a sequence encoding poly-A region and a sequence encoding a 5′ pseudo-cap element capable of mediating translation initiation in the eukaryotic host cell via an internal ribosome entry site (IRES) element.

14. The composition of claim 13, wherein the poly-A region contains 1-500 A's.

15. A system for generating eukaryote-translatable mRNA comprising an engineered bacterium having a sequence encoding a eukaryote-translatable mRNA from the chromosome of the bacterium, wherein transcription of the eukaryote-translatable mRNA is under the control of a promoter that is inactive in a eukaryotic cell and the sequence encoding the eukaryote-translatable mRNA encodes a 5′ IRES and a 3′ poly-A tail.

16. The system for generating eukaryote-translatable mRNA according to claim 15 wherein the promoter is a prokaryotic promoter.

17. The system for generating eukaryote-translatable mRNA according to claim 15 wherein the bacterium is a non-pathogenic invasive bacterium.

18. The system for generating eukaryote-translatable mRNA according to claim 15 wherein the bacterium is a nonpathogenic bacterium that has been engineered to have at least one invasion factor to facilitate entry into a eukaryotic cell or release from a eukaryotic cell endosome.

19. A system for generating eukaryote-translatable SARS-CoV-2 (or other coronavirus) mRNA encoding a viral protein comprising a bacterium having at least one expression cassette comprising a sequence encoding a 5′ IRES and a sequence encoding a eukaryote-translatable mRNA for a coronavirus polypeptide or fragment thereof, wherein transcription of the sequence encoding the eukaryote-translatable mRNA is under the control of a promoter that is inactive in a eukaryotic cell.

20. The system for generating eukaryote-translatable mRNA according to claim 19 wherein the bacterium is a non-pathogenic invasive bacterium.

21. The system for generating eukaryote-translatable mRNA according to claim 19 wherein the bacterium is a nonpathogenic bacterium that has been engineered to have at least one invasion factor to facilitate entry into a eukaryotic cell or release from a eukaryotic cell endosome.

22. The system for generating eukaryote-translatable mRNA according to claim 21 wherein the invasion factor is encoded by an inv or hlyA gene.

23. The system for generating eukaryote-translatable mRNA according to claim 19 wherein the promoter is a prokaryotic promoter.

24. (canceled)

25. (canceled)

26. A method for treating or preventing disease in a subject comprising the step of administering to the host a composition comprising the system of claim 4.

27. The method according to claim 26 wherein the composition is delivered by intramuscular or intranasal administration.

28. The system for generating eukaryote-translatable mRNA according to claim 1 wherein the encoded 5′ pseudo-cap element is an encoded 5′ IRES and the nucleic acid sequence encoding a polypeptide is a sequence encoding a eukaryote-translatable mRNA for viral polypeptide or fragment thereof.

29. The system for generating eukaryote-translatable mRNA according to claim 28 wherein the viral polypeptide or fragment thereof is from a virus listed in Table 2.

30. The system for generating eukaryote-translatable SARS-CoV-2 (or other coronavirus) mRNA encoding a viral protein according to claim 19 wherein the sequence encoding a eukaryote-translatable mRNA for a coronavirus polypeptide or fragment thereof is a sequence encoding a eukaryote-translatable mRNA for a SARS-CoV-2 polypeptide or fragment thereof.
